(19) United States
(12) Patent Application Publication
(10) Pub. No.: US 2026/0368884 A1
(43) Pub. Date: Nov. 19, 2026

(54) SYSTEMS AND METHODS FOR DETECTION, SEGMENTATION AND VISUALIZATION OF ABNORMAL TISSUE REGIONS IN MEDICAL IMAGES

(71) Applicant: SUNNYBROOK RESEARCH INSTITUTE, Toronto (CA)

(72) Inventors: Chinthaka HEYN, Toronto (CA); Hany SOLIMAN, Etobicoke (CA); Alan R. MOODY, Toronto (CA); Jonathan Eric BISHOP, Toronto (CA)

(21) Appl. No.: 19/142,485

(22) PCT Filed: Dec. 20, 2023
(86) PCT No.: PCT/CA2023/051720

§ 371 Date: Jun. 23, 2025

Related U.S. Application Data
(60) Provisional application No. 63/435,139, filed on Dec. 23, 2022.
(60) Provisional application No. 63/537,092, filed on Sep. 7, 2023.

(51) Int. Cl. G06T 7/00; G06T 7/11; G06T 7/32

(52) U.S. Cl. CPC G06T 7/0012; G06T 7/11; G06T 7/32; G06T 2207/10081; G06T 2207/10088; G06T 2207/10104; G06T 2207/20081; G06T 2207/30016; G06T 2207/30096

(57) ABSTRACT

Systems and methods are disclosed that employ an indirect exclusionary approach to the identification of abnormal tissue regions based on the processing of a plurality of multimodal and/or multiparametric image datasets having respective parameters defining a parameter space. A region of parameter space associated with normal tissue, optionally determined in a subject-specific manner, is employed to facilitate the indirect detection and identification of voxels and/or regions associated with abnormal tissue. In some example embodiments, additional parameter space criteria may be employed to further limit the voxels associated with abnormal tissue. A physical space image showing locations of abnormal tissue voxels, or segmented regions associated therewith, may be presented and optionally dynamically updated as additional parameter space criteria is varied in response to user input. Abnormal tissue voxels may be detected locally or throughout an organ. Segmented regions may be employed for the training of a machine learning algorithm.

### CROSS-REFERENCE TO RELATED APPLICATION

This application claims priority to U.S. Provisional Patent Application No. 63/435,139, titled “SYSTEMS AND METHODS FOR DETECTION, SEGMENTATION AND VISUALIZATION OF ABNORMAL TISSUE REGIONS IN MEDICAL IMAGES” and filed on Dec. 23, 2022, and claims priority to U.S. Provisional Patent Application No. 63/537,092, titled “SYSTEMS AND METHODS FOR DETECTION, SEGMENTATION AND VISUALIZATION OF ABNORMAL TISSUE REGIONS IN MEDICAL IMAGES” and filed on Sep. 7, 2023, the entire contents of which are incorporated herein by reference.

### BACKGROUND

The present disclosure relates to the processing of medical images. More particularly, the present disclosure relates to methods of identification and segmentation of abnormal tissue regions in medical images.

Image segmentation is an important first step for the characterization, diagnosis and treatment of diseases using medical images. For example, image segmentation can be applied in the field of neuro-oncology where quantifying brain tumor size (dimensions or volume) at baseline and then at follow-up is part of routine clinical practice and important for determining response to treatment. Following segmentation of the target lesion, measurement of brain tumor signal characteristics such as tumor water diffusion, blood flow or radiomics features are currently being investigated as alternative ways of predicting or evaluating response to treatment for example.

Segmentation methods can be broadly categorized into conventional segmentation algorithms such as those based on thresholding, region-based, fuzzy theory or edge-detection or algorithms based on machine learning, such as deep learning. The development of machine learning models has traditionally required large datasets of segmented lesions which is both time consuming and costly to implement. In addition, the use of trained machine learning models across different centers further requires additional training on local data representing another barrier to their implementation.

One of the advantages of conventional segmentation algorithms is that they do not require a priori training and can be implemented with greater ease. Unfortunately, conventional segmentation algorithms suffer from a number of problems. For example, methods based on thresholding have difficulties handling lesions with complex features and can have lower specificity. Region-based algorithms such as watershed or region-growing are noise sensitive and suffer from poor performance for images with high levels of noise. Other techniques including fuzzy theory algorithms, such as fuzzy c-means clustering, are computationally intensive. Additionally, all conventional segmentation algorithms rely on a user to determine what is abnormal, and should be included in a segmentation, and what is normal and should be excluded.

### SUMMARY

Systems and methods are disclosed that employ an indirect exclusionary approach to the identification of abnormal tissue regions based on the processing of a plurality of multimodal and/or multiparametric image datasets having respective parameters defining a parameter space. A region of parameter space associated with normal tissue, optionally determined in a subject-specific manner, is employed to facilitate the indirect detection and identification of voxels and/or regions associated with abnormal tissue. In some example embodiments, additional parameter space criteria may be employed to further limit the voxels associated with abnormal tissue. A physical space image showing locations of abnormal tissue voxels, or segmented regions associated therewith, may be presented and optionally dynamically updated as additional parameter space criteria is varied in response to user input. Abnormal tissue voxels may be detected locally or throughout an organ. Segmented regions may be employed for the training of a machine learning algorithm.

Accordingly, in a first aspect, there is provided a method of processing image data to identify abnormal tissue, the method comprising:
obtaining a plurality of co-registered image datasets associated with a subject, each image dataset associating values of a respective image parameter with a set of voxels, the respective image parameters of the plurality of co-registered image datasets defining a parameter space;
employing a clustering algorithm in the parameter space to:
   select a cluster of voxels associated with normal tissue; and determine properties of a distribution associated with the selected normal tissue cluster;
employing properties of the distribution to determine parameter space criteria suitable for identifying abnormal tissue voxels; and
processing the image datasets to identify, from a selected set of voxels, an abnormal set of voxels having image parameters satisfying the parameter space criteria.

In some example implementations of the method, the method further comprises generating an image that facilitates identification, in physical space, of locations at least a portion of the abnormal set of voxels. The method may further comprise defining, within the parameter space, a normal tissue parameter space region based on statistical properties of the distribution. The parameter space criteria may be generated based on the normal tissue parameter space region.

In some example implementations of the method, the parameter space criteria comprises one or more thresholds, each threshold being associated with a respective parameter space axis, and wherein each threshold is generated based on a statistical measure associated with the distribution.

In some example implementations of the method, the parameter space criteria comprises a region having boundary values determined based on statistical measures associated with the distribution.

In some example implementations of the method, the image includes normal voxels having image parameters residing within the normal tissue parameter space region, and wherein the abnormal set of voxels are displayed such that they are identifiable relative to the normal voxels. The image may exclude normal voxels having image parameters residing within the normal tissue parameter space region.

In some example implementations of the method, the method further comprises employing a segmentation algorithm to segment at least one contiguous group of voxels within the abnormal set of voxels, thereby obtaining a segmented region. The method may further comprise generating an image that facilitates identification of the segmented region. The method may further comprise employing the segmented region to train a machine learning algorithm. The segmented region may be employed as a seed for a region-growing algorithm. The method may further comprise processing the image to identify a necrotic region and annotating the image within the necrotic region.

In some example implementations of the method, the abnormal set of voxels are identified from voxels corresponding to a single image slice.

In some example implementations of the method, the abnormal set of voxels are identified from voxels corresponding to a plurality of image slices.

In some example implementations of the method, the abnormal set of voxels are identified from voxels spanning an entirety of an organ.

In some example implementations of the method, the parameter space is modified, prior to identifying the abnormal set of voxels, according to one or more statistical measures associated with the parameter values of voxels associated with normal tissue.

In some example implementations of the method, at least one parameter space axis of the parameter space is normalized, prior to identifying the abnormal set of voxels, based on a z-score of voxels associated with normal tissue.

In some example implementations of the method, the parameter space origin is modified, prior to identifying the abnormal set of voxels, based on a centroid associated with voxels associated with normal tissue.

In some example implementations of the method, the parameter space criteria is autonomously applied.

In some example implementations of the method, the parameter space criteria is selectable on a user interface presented to a user.

In some example implementations of the method, the method further comprises applying additional parameter space criteria, prior to obtaining the abnormal set of voxels from the selected set of voxels, such that the abnormal set of voxels are excluded from the normal tissue parameter space region and also satisfy the additional parameter space criteria.

In some example implementations of the method, the method further comprises, after having obtained the abnormal set of voxels, applying additional parameter space criteria to the abnormal set of voxels, thereby obtaining a subset of abnormal voxels that satisfy the additional parameter space criteria. The additional parameter space criteria may be configured such that the subset of abnormal voxels corresponds to tumor enhancing voxels. The parameter space may be an initial parameter space, the method further comprising representing the subset of abnormal voxels in a secondary parameter space that is different from the initial parameter space. The method may further comprise applying a supplementary criterion to the secondary parameter space to differentiate among at least two abnormal tissue types. The additional parameter space criterion may be configured such that the subset of abnormal voxels correspond to enhancing tumor voxels, and wherein the supplementary criterion is configured to facilitate differentiation between voxels associated with tumor progression and voxels associated with radiation necrosis. The supplementary criterion may comprise a T2 FLAIR post Gadolinium signal threshold.

The parameter space criteria may be based on a normal tissue parameter space region defined according to statistical properties of the distribution, the method further comprising:
generating a cluster plot showing:
   locations of the selected set of voxels in the parameter space;
   the normal tissue parameter space region; and
   an annotation representing the additional parameter space criterion;
generating a physical space image showing locations of voxels excluded from the normal tissue parameter space region and satisfying the additional parameter space criterion;
receiving input from the user for adjusting the additional parameter space criterion; and
dynamically updating the physical space image as the additional parameter space criterion is varied by the user.

In some example implementations of the method, the normal tissue parameter space region is determined by processing the plurality of co-registered image datasets, within the subset of voxels associated with normal tissue, such that the normal tissue parameter space region substantially encloses a cluster of normal tissue voxels.

In some example implementations of the method, the normal tissue parameter space region is determined by processing reference image data associated with a set of reference subjects.

In some example implementations of the method, the normal tissue parameter space region is determined, at least in part, according to user input, the user input identifying a subset of the set of voxels that is associated with the presence of normal tissue.

In some example implementations of the method, the set of voxels spans a single image slice identified as containing normal tissue. The set of voxels may span a plurality of image slices identified as containing normal tissue.

In some example implementations of the method, the method further comprises receiving user instructions to dilate the normal tissue parameter space region, and dilating the normal tissue parameter space region according to the user instructions.

In some example implementations of the method, the set of voxels are separated from a larger set of voxels prior to applying the parameter space criteria, the set of voxels pertaining to an anatomical region of interest that resides within the larger set of voxels.

In some example implementations of the method, the image parameter associated with at least one of the image datasets is a signal intensity.

In some example implementations of the method, at least two image datasets of the plurality of co-registered image datasets are associated with different imaging modalities.

In some example implementations of the method, at least two plurality of co-registered image datasets of the plurality of co-registered image datasets are multiparametric image datasets associated with a common imaging modality.

At least two imaging datasets of the multiparametric image datasets may be magnetic resonance imaging datasets. The magnetic resonance imaging datasets may include a T1 MPRAGE post-gadolinium image dataset and a T2FLAIR post-gadolinium image dataset. The magnetic resonance imaging datasets may include a T2 STIR image dataset and a T1 pre-gadolinium image dataset. At least two imaging datasets of the multiparametric image datasets may include CT imaging datasets acquired with different scan voltages.

In some example implementations of the method, at least two of the image datasets are normalized.

In another aspect, there is provided a system for processing image data to identify abnormal tissue, the system comprising:
processing circuitry comprising at least one processor and associated memory, said memory storing instructions executable by said at least one processor for performing operations comprising:
   obtaining a plurality of co-registered image datasets associated with a subject, each image dataset associating values of a respective image parameter with a set of voxels, the respective image parameters of the plurality of co-registered image datasets defining a parameter space;
   employing a clustering algorithm in the parameter space to:
      select a cluster of voxels associated with normal tissue; and determine properties of a distribution associated with the selected normal tissue cluster;
   employing properties of the distribution to determine parameter space criteria suitable for identifying abnormal tissue voxels; and
   processing the image datasets to identify, from a selected set of voxels, an abnormal set of voxels having image parameters satisfying the parameter space criteria.

In another aspect, there is provided a non-transitory computer-readable storage medium having stored therein data representing instructions executable by a processor for processing image data to identify pathology, the storage medium comprising instructions for performing operations including:
obtaining a plurality of co-registered image datasets associated with a subject, each image dataset associating values of a respective image parameter with a set of voxels, the respective image parameters of the plurality of co-registered image datasets defining a parameter space;
employing a clustering algorithm in the parameter space to:
   select a cluster of voxels associated with normal tissue; and determine properties of a distribution associated with the selected normal tissue cluster;
employing properties of the distribution to determine parameter space criteria suitable for identifying abnormal tissue voxels; and
processing the image datasets to identify, from a selected set of voxels, an abnormal set of voxels having image parameters satisfying the parameter space criteria.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 shows an example method for performing pathology-agnostic detection of abnormal tissue based on the processing of multi-parametric and/or multimodal image datasets.

FIG. 2 schematically illustrates the selection, within a parameter space, of a tissue cluster associated with normal tissue, and the determination of properties of a distribution associated with the selected normal tissue cluster.

FIGS. 3A, 3B, 3C and 3D illustrate an example method of identifying voxels associated with abnormal tissue and generating a physical space visualization.

FIGS. 4A and 4B illustrate the application of criteria generated based on properties of the distribution associated with the normal tissue to exclude additional voxels when identifying voxels associated with abnormal tissue.

FIGS. 4C and 4D illustrate an alternative implementation of the application of parameter space criteria generated based on properties of the distribution associated with the normal tissue to exclude voxels when identifying voxels associated with abnormal tissue.

FIG. 5 is a flow chart illustrating dynamic user selection and application of additional criteria to exclude additional voxels when identifying voxels associated with abnormal tissue.

FIG. 6 schematically presents an example system for performing pathology-agnostic detection of abnormal tissue based on the processing of multi-parametric and/or multimodal image datasets.

FIG. 7 is a flow-chart presenting an example application involving the detection and segmentation of brain metastases FIG. 8 shows images demonstrating application of the example methods of the present disclosure to the detection and segmentation of brain metastases.

FIGS. 9A and 9B show co-registered MR data for two different pulse sequences, namely T2FLAIR post-gad and T1MPRAGE post-gad.

FIGS. 9C and 9D illustrate the determination of a normal tissue parameter space based on the processing of a slice that is known or expected to include normal tissue.

FIGS. 10A, 10B, 10C and 10D illustrate the application of the normal tissue parameter space, and additional parameter space criteria, to identify voxels associated with abnormal tissue.

FIGS. 11A and 11B demonstrate the performance of the example method for the detection of brain metastases when compared against the gold standard of manual segmentation.

FIGS. 12A, 12B and 12C demonstrate blood vessel exclusion on tumor segmentation using the application of additional parameter space criteria.

FIGS. 13A, 13B, 13C, and 13D show steps for generating a normalized parameter space based on parameter values of normal tissue.

FIGS. 14A, 14B, 14C, 14D, 14E and 14F demonstrate the segmentation of abnormal tissue voxels, based on the normalized parameter space, within an image slice containing abnormal tissue.

FIGS. 14G and 14H show application of thresholds in the normalized parameter space and resulting segmentation containing normal and abnormal tissue.

FIGS. 15A, 15B and 15C illustrate segmentation of enhancing tumor according to varying example T1 MPRAGE thresholds.

FIGS. 16A, 16B, 16C, 16D and 16E illustrate the identification of tumor progression voxels, among the tumor enhancing voxels, based on additional thresholds applied to tumor enhancing voxels having T2 FLAIR post Gadolinium signal exceeding a selected additional threshold.

FIG. 17 illustrates an example workflow that permits the selection, in an intermediate physical image representation generated after initial parameter-spaced-based removal of normal tissue voxels, of different tissues for inclusion and/or removal in the final physical image space segmentation, where the inclusion or exclusion is performed based on the clustering of voxels associated with the different tissue types in parameter space.

FIG. 18 shows an alternative workflow, relative to that shown in FIG. 17, in which a region growing step is performed in the intermediate physical image representation to facilitate the selection of the different tissues for inclusion and/or removal in the final physical image space segmentation.

FIG. 19 shows an example workflow in which a parameter-space-based processing method is employed to selectively identify voxels corresponding to a selected tissue type in a multimodal or multiparametric image dataset, and to employ these identified voxels to modify the display of the selected tissue types in one of the images forming of the multimodal or multiparametric dataset, for example, to darken or otherwise modify the visibility of the tissue type.

FIGS. 20A and 20B illustrate how the method illustrated in FIG. 19 can improve the detectability of small metastases next to blood vessels. An original T1 MPRAGE post-Gd image is shown in FIG. 20A, while FIG. 20B shows the blood vessel subtracted image.

FIG. 21 illustrates an example processing method in which two dimensions of the parameter space are associated co-registered image data that is acquired at different imaging sessions, and where each of the two dimensions is associated with the same imaging parameter, thereby enabling the detection of changes in temporal progression of abnormal tissue.

FIG. 22 shows an example workflow for use in tandem with a deep learning algorithm in order assess the accuracy of the deep learning algorithm or to edit the segmentation produced by the deep learning algorithm.

FIG. 23 plots volume of non-enhancing tumor for a patient with a low grade glioma on treatment measured using deep learning (nnUnet) and the present invention.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As used herein, the term “criteria” refers to one or more criterion.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the phrase “system acquisition parameter” refers to a parameter employed to control the acquisition of image data. An example of a system acquisition parameter is a sequence parameter of an MRI system, such as B₀, TE, TR and flip angle. Another example of a system acquisition parameter is the voltage of a CT scanner.

As used herein, the phrase “image parameter” refers to a signal value, or a parameter obtained via processing of a signal value, that is dependent on one or more system acquisition parameters. Examples of image parameters are signal values in MRI, which depend on various system acquisition parameters of an MRI sequence and properties of an MRI scanner, and the CT number in CT imaging, which is dependent, for example, on scan voltage and CT system vendor. An image parameter may be generated by processing and combining two or more other image parameters, in order to obtain an image parameter characterizing a lower dimensional space.

As used herein, the phrase “intrinsic physical tissue parameter” refers to a measure of an intrinsic physical property of tissue that is independent of a given imaging modality. An example of an intrinsic physical tissue parameter is a physical parameter of an MRI image, such as T1, T2 and T2*.

As used herein, the phrase “quantitative MRI” (qMRI) refers to the measurement of one or more intrinsic physical tissue parameters by performing multiple MRI acquisitions while varying one or more acquisition parameters, and employing an inverse model to determine, on a per-voxel basis, one or more intrinsic physical tissue parameters.

As used herein, the phrase “MR fingerprinting” (MRF) refers to a method that permits the measurement of multiple intrinsic physical tissue parameters using a dictionary fitting approach. MRF can be understood as a three-step process that includes (i) data acquisition, (ii) pattern matching to a dictionary and (iii) tissue property determination and visualization. MRF data acquisition occurs while performing an MRF sequence in which MR acquisition parameters are varied, in a pseudorandom manner, in order to generate unique signal evolutions, or “fingerprints”. The fingerprints from individual voxels, for a given MRF sequence, are compared with a collection of simulated fingerprints contained in a dictionary correlating previously measured fingerprints with known intrinsic physical tissue parameters. The best match for the voxel fingerprint is selected from the dictionary through a pattern matching process. Once a pattern match is detected, the combination of intrinsic physical tissue parameters associated with the matched pattern are identified as the underlying intrinsic physical tissue parameters in the voxel, thereby facilitating quantitative and anatomic maps of intrinsic physical tissue parameters. MRF can be employed, for example, to infer intrinsic physical tissue parameters such as T1, T2, static magnetic field (B₀) inhomogeneity or off-resonance frequency and proton density M0, among other properties.

As used herein, the phrase “abnormal tissue” refers to tissue containing pathology.

As used herein, the phrases “normal tissue” and “healthy tissue” refer to tissue absent of pathology.

As used herein, the phrase “imaging modality” broadly refers to an imaging technology that gathers, generates, processes and/or analyzes imaging information of a target body through a particular detection of transduction mechanism. Non-limiting examples of imaging modalities include x-ray, computed x-ray tomography (also called computed tomography and abbreviated as CT), magnetic resonance imaging (MRI), ultrasound imaging, positron emission tomography (PET), single photon emission computed tomography (SPECT), optical tomography and fluorescence imaging.

As used herein, the phrase “multimodal imaging” or “multiple imaging modalities” refers to the collection of multiple image datasets using at least two imaging modalities.

As used herein, the phrase “multiparametric” refers to the use of a common imaging modality with different system acquisition parameters to collect multiple image datasets. For example, multiparametric MRI refers to the collection of multiple MRI image datasets using different pulse sequences.

As used herein, the term “voxel” refers to an element within a volume. Although various embodiments disclosed herein involve voxels that define three-dimensional elements within a three-dimensional volume, it will be understood that some embodiments may involve image data associated with a two-dimensional portion of a volumetric region, and in such cases the two-dimensional portion of the volumetric region may characterized by two-dimensional voxels. For example, image data characterizing a two-dimensional slice may be represented in the form of voxels having a two-dimensional shape, and the slice may be further characterized by a thickness.

In view of the aforementioned challenges and limitations of conventional methods of image segmentation, the present inventors sought out to develop a rapid and efficient method for detecting and segmenting abnormal tissue regions from medical image data. In contrast to conventional methods and algorithms that rely on the direct identification of abnormal tissue characteristics in image data to achieve segmentation, various example embodiments of the present disclosure employ an orthogonal and indirect approach that does not rely on the direct identification of voxels associated with abnormal tissue based on known or expected properties of abnormal tissue. Instead, various embodiments of the present disclosure employ “pathology agnostic” methods in which abnormal tissue is indirectly identified, on an exclusionary basis, by identifying voxels that do not exhibit properties associated with normal tissue. Such embodiments, which employ the properties of normal tissue to perform segmentation and detection, without requiring any a priori knowledge or assumptions regarding the pathology, may provide a more robust and efficient approach to the detection and segmentation of abnormal tissue.

As will be described in further detail below, the present example embodiments may be implemented in the absence of a priori training and facilitate the identification of abnormal tissue by differentiating it from normal tissue in an objective manner. The end result is an improvement in specificity compared to previously described conventional algorithms.

Various example embodiments of the present disclosure employ an indirect exclusionary approach to the identification of abnormal tissue regions based on the processing of a plurality of multimodal and/or multiparametric image datasets having an associated parameter space. A distribution associated with normal tissue within the parameter space is employed to facilitate the indirect detection and identification of voxels and/or regions associated with abnormal tissue. A (statistical) distribution, as used herein, refers to a function which shows possible values for a variable and how often they occur. Signal intensities of a tissue form a distribution of values which depend on the composition of the tissue and characteristics of the imaging modality such as electronic noise. A simple tissue signal distribution may be Gaussian or can be modelled as a Gaussian distribution. More complex tissues may be more complex, for example bimodal or multimodal.

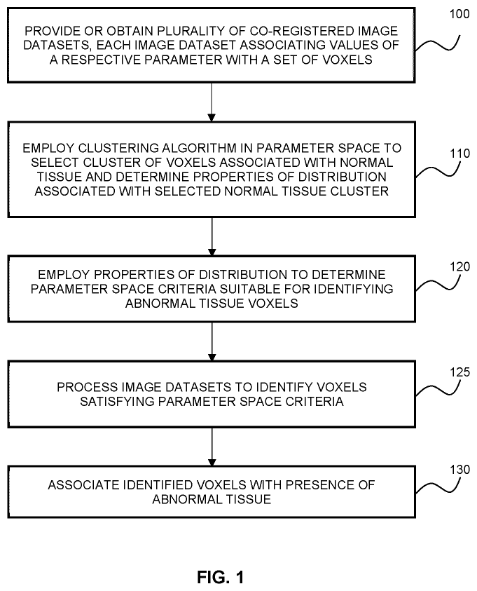

FIG. 1 provides a flow chart that illustrates an example method of indirect detection of abnormal tissue in multimodal and/or multiparametric image datasets associated with a subject. As shown at step 100, a plurality of image datasets are provided, with each image dataset associating values of a respective parameter with a set of voxels, where the voxels span an anatomical region of interest, such as the brain. If not already provided in spatially co-registered form, the image datasets are processed to perform co-registration. Many different methods of image co-registration will be known to those skilled in the art, and non-limiting examples of such methods include rigid, affine and deformable registration algorithms.

The parameters of the plurality of image datasets define a multi-dimensional parameter space. In some example implementations, the parameter space is a low dimensional parameter space that includes 2 or 3 dimensions; however, other implementations may include 4 or more parametric dimensions. As described in further detail below, the imaging datasets, and their associated parameters, are selected such that voxels corresponding to normal tissue cluster together in parameter space without substantial overlap with voxels associated with one or more pathologies of interest. Signal intensities for a tissue will show a range of values with mean, median, mode and standard deviation. At step 110, a clustering algorithm in the parameter space to select a cluster of voxels associated with normal tissue, and to determine properties of a distribution associated with the selected normal tissue cluster.

In step 120, properties of the normal tissue distribution are employed to determine parameter space criteria suitable for identifying abnormal tissue voxels. In some example implementations, this criteria may delineate (enclose) a parameter space region that is associated with normal tissue, and this region is henceforth referred to as the normal tissue parameter space region. As described below, the parameter space criteria may be determined by processing the image datasets within a subset of voxels that are known or expected to be associated with normal tissue. The plurality of image datasets are processed in step 125, according to the parameter space criteria, to identify voxels that are associated with abnormal tissue. The identified voxels, which may be further limited based on additional criteria within the parameter space, may be associated with abnormal tissue, as shown at step 130. As will be described in further detail below, the abnormal tissue voxels may be processed to generate one or more segmented abnormal tissue regions.

The selected cluster of normal tissue voxels and associated normal tissue distribution, which are defined in step 110 of FIG. 1, may be defined or determined according to many different implementations. For example, in some implementations, the selected cluster of normal tissue voxels and associated normal tissue distribution may be determined based on processing reference image datasets pertaining to a set of subjects exhibiting normal tissue.

In other example implementations, the selected cluster of normal tissue voxels and associated normal tissue distribution may be defined or determined based on the plurality of image datasets associated with the subject. Such a subject-specific (subject-customized) determination of the normal tissue parameter space may be performed, for example, by first determining or identifying a subset of voxels associated with normal tissue of the subject, and then employing the subset of voxels to determine the selected cluster of normal tissue voxels and associated normal tissue distribution. The present inventors have found that the use of subject-specific imaging datasets to characterize normal tissue and to define the selected cluster of normal tissue voxels and associated normal tissue distribution may yield more robust and accurate detection of abnormal tissue.

The subset of voxels may be defined, for example, in one or more two-dimensional slices that are known or expected to exhibit normal tissue. For example, a user interface may be employed by a user to view multiple image slices showing image data from one or more of the imaging datasets (or one or more other co-registered image datasets), which may be useful in enabling the user to identify one or more slices that are known or expected to be associated with normal tissue. The user may then provide input selecting the one or more image slices (or one or more regions within one or more slices) that are known or expected to be associated with normal tissue. In some cases, a user may select, from one or more image slices, at least one subregion of voxels that is known or expected to be associated with normal tissue. At least one subregion of voxels that is known or expected to be associated with normal tissue may be defined, for example, by via a user interface, or, for example, by a machine learning algorithm. For example, in the latter case, a deep learning algorithm may be employed to identify normal white matter and/or normal grey matter.

In some example implementations, voxels associated with one or more known anatomical structures may be removed prior to determining the normal tissue parameter space region. For example, in some cases where the imaging datasets pertain to the head, the voxels associated with brain tissue may be separated (e.g. extracted) from voxels associated with other cranial structures, and the normal tissue parameter space region may be determined based on processing the subset of voxels associated with brain tissue. Such an organ extraction step may be performed, for example, using conventional extraction methods (e.g. thresholding) or using machine learning approaches such as deep learning.

In some example implementations, the subset of voxels employed to determine the selected cluster of normal tissue voxels and associated normal tissue distribution may be associated with a subset of tissue types that are present within an anatomical region of interest. For example, the subset of voxels employed to determine the selected cluster of normal tissue voxels and associated normal tissue distribution may only include grey matter or white matter, or may predominately include (i.e. containing at least 80%, 85%, 90% or 95% of) grey matter or may predominately include white matter.

In some example implementations, the subset of tissue types, from which the subset of voxels utilized to determine the selected cluster of normal tissue voxels and associated normal tissue distribution, may exclude a tissue type that represents a majority of the tissue voxels within the image dataset. In some example implementations, the subset of tissue types, from which the subset of voxels utilized to determine the selected cluster of normal tissue voxels and associated normal tissue distribution, may represent a minority of the tissue voxels within the image dataset. For example, the subset of voxels utilized to determine the selected cluster of normal tissue voxels and associated normal tissue distribution may be limited to cerebral spinal fluid.

It will be understood that the image data that is processed to identify the selected cluster of normal tissue voxels and associated normal tissue distribution need not exclusively contain normal tissue voxels. For example, one or more image datasets that are processed to identify the normal tissue parameter space may include one or more abnormal tissue voxels, provided that the abnormal tissue voxels are present in a sufficiently low fraction, relative to the normal tissue voxels, to permit identification of the selected cluster of normal tissue voxels and associated normal tissue distribution. It will be understood that the determination of whether a sufficiently low fraction of abnormal tissue voxels is present within an image dataset to facilitate the determination of the normal tissue parameter space may vary based on the clinical context, including, for example, the type of pathology, the imaging modalities selected, and the size of the total image volume relative to an expected volume of abnormal tissue. In some example cases in which abnormal tissue voxels are expected or assumed (e.g. based on a preliminary assessment of image data) to make up a sufficient small percentage of the total imaging volume to permit identification of the selected cluster of normal tissue voxels and associated normal tissue distribution, the voxels from the total imaging volume may be processed to identify the selected cluster of normal tissue voxels and associated normal tissue distribution.

Having identified a subset of voxels that are known or expected to be associated with normal tissue of the subject, the values of the parameters associated with these voxels (as per the multiple imaging datasets) may be employed to determine the selected cluster of normal tissue voxels and associated normal tissue distribution within which voxels associated with normal tissue are clustered. In some example implementations, this step may be performed in an automated manner.

For example, the selected cluster of normal tissue voxels and associated normal tissue distribution may be determined based on one or more clustering algorithms including centroid-based (e.g. k-means clustering, fuzzy c-means), density-based or distribution-based methods. Alternatively, the selected cluster of normal tissue voxels and associated normal tissue distribution may be identified using deep learning methods.

In other example implementations, the selected cluster of normal tissue voxels and associated normal tissue distribution may be determined based on input from a user. For example, a scatter plot, or other suitable visualization, may be employed to facilitate a visual representation of the locations of the normal tissue voxels within the parameter space, and input from a user can be received that defines a region in the parameter space that is associated with clustering of these voxels, from which a distribution can be computed.

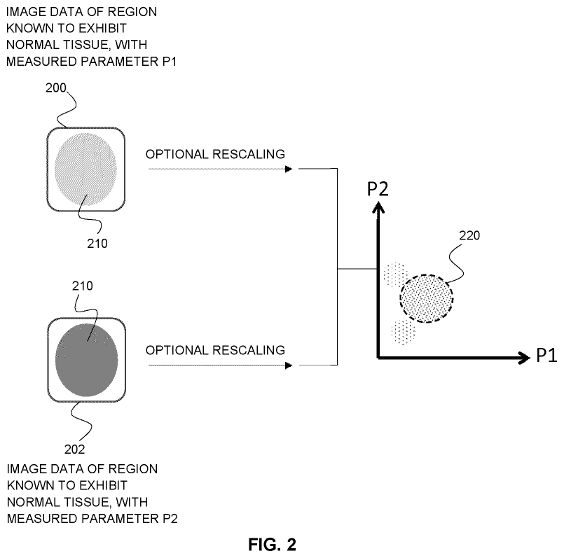

An example method of the determination of the selected cluster of normal tissue voxels and associated normal tissue distribution is illustrated in FIG. 2. The figure illustrates an example implementation involving two image datasets, one providing per-voxel values of a first parameter P1, and another providing per-voxel values of a first parameter P2. The two image datasets are spatially co-registered.

While the first and second imaging datasets are volumetric image datasets, FIG. 2 illustrates step 110 of FIG. 1, in which a subset of voxels within an image slice are identified as being known or expected to pertain to normal tissue, shown as images 200 and 202. These images respectfully correspond to the first and second image datasets associated with the subject within the selected image slice. In some example implementations, a subregion of voxels 210 may be selected within the image slice as pertaining to normal tissue, for example, by reference to one or more of the images 200 and 202 of the slice.

As shown in FIG. 2, the voxels identified as being associated with normal tissue are employed to select a cluster of normal tissue voxels and determine an associated normal tissue distribution in the parameter space spanned by P1 and P2, within which normal tissue voxels cluster. This step is optionally performed after first rescaling the parameters P1 and P2, as shown in the figure. A normal tissue parameter space region characterizing the selected cluster of normal tissue voxels and associated normal tissue distribution is shown at 220. This normal tissue parameter space, and/or other parameter space criteria associated with the selected cluster of normal tissue voxels, may be determined by processing the distribution of parameter values associated with the selected cluster to determine a suitable bounding region that predominantly or substantially encloses clustered normal tissue voxels. For example, the parameter space boundary defining the normal tissue parameter space region may be defined by a selected percentile (e.g. the 99th percentile) of values, resulting in the region demarcated by the dashed ovoid in FIG. 2. In the present example implementation, the normal tissue parameter space region 220 is determined based on subject-specific image data. The normal tissue within the dashed ovoid correspond to a combination of grey and white matter voxels. Additional smaller normal tissue clusters corresponding to CSF (lower left) and enhancing blood vessels (upper left) are also illustrated in FIG. 2.

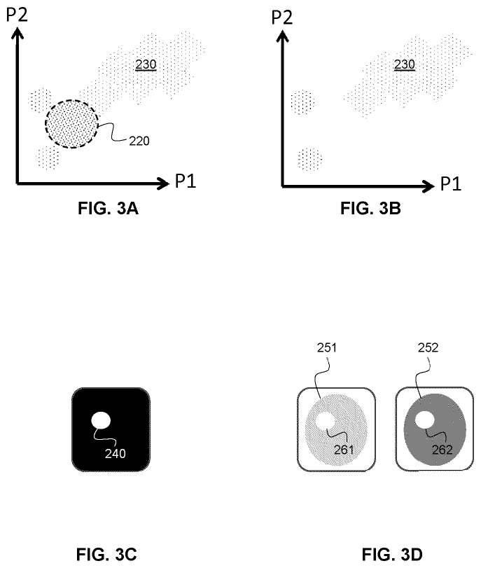

Having selected the cluster of normal tissue voxels and determined the associated normal tissue distribution, one or more properties of the normal tissue distribution may be employed to generate parameter space criteria, which, when satisfied, may be indicative of abnormal tissue, and this parameter space criteria can be employed to identify abnormal tissue in other voxels, such as another image slice or over the entity of the set of voxels. This step is illustrated in FIGS. 3A and 3B. FIG. 3A shows a cluster plot that includes voxels residing within a different slice than the slice that had been employed to define the normal tissue parameter space (e.g. a different slice that may include abnormal tissue). A healthy parameter space region, computed based on the properties of the previously determined normal tissue distribution, is shown at 220 and voxels 230 residing outside of the region 220 are identified as being associated with abnormal tissue. FIG. 3B shows a cluster plot that only includes the voxels 230 residing outside of the normal tissue parameter space region 220 after additional parameter space criteria associated with the normal tissue distribution is applied, schematically illustrating the exclusionary identification of abnormal tissue voxels based on features associated with normal tissue.

In some example implementations, a data structure representing the subset of voxels associated with abnormal tissue (or being suspected of being abnormal tissue) may be obtained by removing (subtracting)the set of voxels that fail to satisfy the parameter space criteria (for example, in the non-limiting case of criteria defining a normal tissue parameter space region enclosed by an ellipse or ellipsoid centered on the normal cluster and having an extent defined, in each parameter space dimension, based on a standard deviation associated with normal tissue cells of the selected cluster, the voxels within the ellipsoid), leaving behind the residual voxels associated with abnormal tissue. Alternatively, a separate data structure representing the subset of voxels associated with abnormal tissue (or being suspected of being abnormal tissue) may be generated from the voxels that satisfy the parameter space criteria (e.g. in the case of the aforementioned normal parameter space region defined by an ellipse or ellipsoid, the voxels that reside beyond the normal tissue parameter space region, optionally applying one or more additional thresholds associated with the normal tissue distribution, in one or more parameters to isolate the abnormal voxels).

In some example implementations, prior to employing the properties of the normal tissue distribution to identify abnormal tissue in other voxels, the parameter space may be modified based on one or more statistical measures associated with parameter values of the normal tissue voxels residing within the normal tissue parameter space region. Example modifications include shifting the origin of the parameter space and/or rescaling, e.g. normalizing, the parameters (i.e. the parameter axes). For example, the centroid of the normal tissue cluster may be determined (for example, using a centroid-based algorithm such as K-means clustering, or other clustering algorithms such as Density-based clustering or Distribution-based algorithms), and the modified parameter space may be constructed such that its origin corresponds to this centroid. Additionally or alternatively, for at least one parameter (i.e. one axis) of the parameter space, the parameter values may be rescaled (e.g. normalized) based on one or more statistical measures associated with the normal tissue distribution. For example, one or more parameters defining an initial parameter space may be normalized based on the standard deviation and/or mean of corresponding parameter values of the normal tissue voxels. One or more of the statistical measures may be normalized prior to the measures being employed to scale the parameter space. For example, the standard deviation of the distribution of initial parameter values associated with the normal tissue distribution may be scaled to unity.

In one example embodiment, at least one parameter that was employed to generate an initial parameter space is rescaled according to a z-score computed based on the statistics (respectively associated with the parameter) of the normal tissue distribution, thereby forming a modified parameter space (e.g. such that at least one axis of the modified parameter space represents a respective z-score based on the corresponding statistics of the values of the normal tissue voxels).

For example, in one non-limiting method, an initial parameter space is determined based on parameters associated with co-registered image datasets, and after optionally first performing organ extraction, bias correction, and parameter rescaling, a clustering algorithm such as k-means is employed to determine the normal tissue cluster and associated distribution. The centroid (mean) and cluster standard deviation associated with the normal tissue distribution are used to generate a new modified (standardized) parameter space according to the following transformation for at least one parameter of the initial parameter space: (S-S_{normal_mean})/SDₙₒᵣₘₐₗ where S is the signal intensity in the initial parameter space for a given parameter, S_{normal_mean} is the mean signal intensity of the normal tissue voxels within the normal tissue parameter space for the given parameter, and SDₙₒᵣₘₐₗ is the standard deviation of the voxels within the normal tissue cluster.

While the present disclosure employs a centroid, mean, standard deviation as examples of statistical measures and the z-score as an example of a normalization method/transformation that is based on the statistics of the values of the normal tissue voxels, it will be understood that other statistical measures and transformations may be employed in the alternative. For example, if the tissue voxels of the selected cluster do not follow a sufficiently normal distribution, a Box-Cox transformation may be employed to correct this. Alternatively it may be useful to transform the parameter space with cluster median and median absolute deviation in place of S_{normal_mean} and SD_{normal.}. In contrast, if cluster voxels are normally distributed, it may be preferable deliberately to shift to a non-normal distribution by histogram equalization or similar methods.

The present example embodiment, in which the axes of the parameter space are configured according to statistical measures associated with the normal tissue voxels, may be beneficial in many applications that involve variations in imaging. For example, signal intensities (“image parameters”) for a given imaging modality and/or protocol (e.g. T2 FLAIR acquired with a given set acquisition parameters) can vary from scan to scan, scanner to scanner and day to day. One advantage of employing a parameter space representation that is modified (customized, e.g. normalized) according to the statistical measures associated with the normal tissue voxels is that the modification provides a frame of reference that facilitates an improved comparison between scans. Such an approach enables a comparison longitudinally in a given patient, and possibly between patients scanned on different scanners at different institutions.

The aforementioned approaches to modifying (redefining) the parameter space based on one or more statistical measures associated with the normal tissue distribution can be beneficial because these approaches can move arbitrary signal intensities into a frame of reference which is standardized and enables the determination of pathology as having a signal intensities that are statistically different from background (either higher or lower).

The modification of the parameter space to a standardized parameter space may also be employed to enable a user to statistically visualize abnormal tissues outside of the distribution of normal background tissue. For example, abnormal signal intensities above a certain z-score threshold along one or more parameter space dimensions can be visualized. These thresholds could also be used to define what is abnormal. For example, enhancing disease as any voxel with a signal intensity above a certain threshold. Another advantage of the use of a standardized parameter space is that such a modified parameter space allows a comparison of signal intensities for pathology between patients scanned using similar scanning parameters. Yet another advantage is that a standardized parameter space can improve the speed and accuracy of segmentations using thresholds. For example, an abnormality may typically have a signal intensity above a threshold level on a baseline scan and may have a similar signal intensity and threshold on a follow up scan.

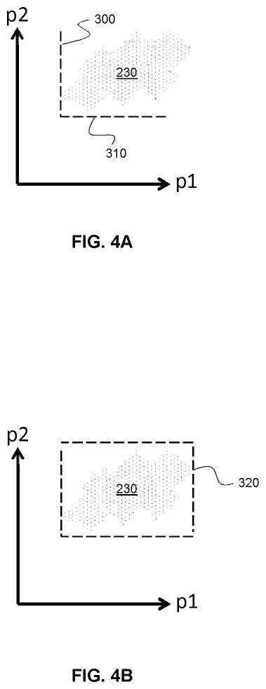

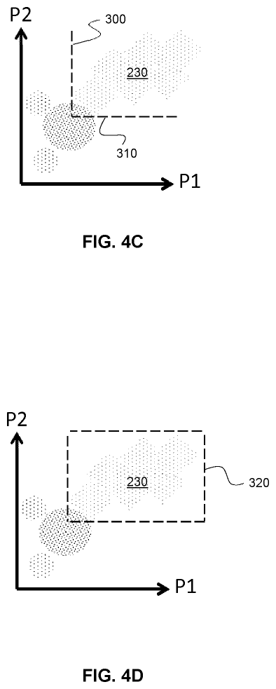

Having identified the subset of voxels associated with abnormal tissue, this subset of voxels may be employed to generate a visualization indicative of the locations of abnormal tissue in physical space. For example, FIG. 3C shows a physical space representation of the image slice processed to identify the abnormal voxels shown at 230 in FIG. 3B. The example image in FIG. 3C displays only the locations of the abnormal voxels at 240 (based on the workflow illustrated in FIGS. 4A and 4B). In some example implementations, the physical space visualization may show the locations of normal and abnormal voxels (including clusters of normal voxels other than those selected to determine the normal tissue distribution), such that the different types of voxels are discernable (e.g. via different color, shading, texture, transparency, or other visual features). In some example implementations, the visualization may also plot one or more parameters, such as parameters associated with one or more of the image datasets. For example, in FIG. 3D, physical space images of the voxel slice are generated showing the spatial mapping of the first and second image parameters P1 and P2 as well as contours showing the regions of normal (251, 252) and abnormal (261, 262) voxels (but excluding the additional normal clusters shown in FIGS. 3A and 3B).

In some example implementations, a segmentation algorithm may be employed to segment, in the physical space representation, at least one contiguous group of voxels within the subset of voxels identified as abnormal tissue, thereby obtaining at least one segmented abnormal tissue region. In some example implementations, a margin may be added to one or more segmented regions. For example, this can be achieved by performing a binary dilation of the segmentation using a structuring element. In some example implementations, one or more regions corresponding to areas of necrosis may be annotated (e.g. “filled in”) when generating a visualization showing the locations of abnormal tissue voxels or segmented regions of abnormal tissue. This can be achieved, for example, by performing a binary dilation of the segmentation using a structuring element followed by a binary erosion using the structuring element. If a closed but narrow contour results from a preliminary segmentation, a flood fill algorithm may be used to finish the segmentation. If a narrow and open contour results from a preliminary segmentation, and a dilation operation is first used to close that contour, then the flood fill can be performed before conducting the matching erosion. If a narrow and open contour cannot be closed with a dilation operation, an active contouring approach with an elastic energy constraint is a suitable match for abnormal tissues that are compact and convex in three-dimensional shape.

In some example implementations, the detection of abnormal tissue voxels, and the optional segmentation of one or more contiguous group of abnormal tissue voxels, may be performed within one selected subregion of the overall set of voxels associated with the imaging datasets (e.g. one image slice), or two or more subregions of the overall set of voxels (e.g. multiple image slices). In some example implementations, the detection of abnormal tissue voxels, and the optional segmentation of one or more contiguous group of abnormal tissue voxels, may be performed within all of the voxels (i.e. the entire volume) associated with the imaging datasets, thereby enabling the identification and optional display of all abnormal tissue within the imaging volume, allowing both simultaneous whole organ detection and segmentation of pathology.

In some example implementations, at least one of the image datasets may be bias corrected prior to defining the normal tissue parameter space region and/or prior to identifying voxels excluded from the healthy parameter space region. For example, bias correction can be beneficial in MR datasets where a low spatial frequency variation in signal intensity occurs within an image as a result of variations in the sensitivity of radiofrequency coils used.

In the present example embodiments, the detection of abnormal tissue voxels may be facilitated by the selection of imaging modalities and associated parameters that result in a cluster of normal tissue voxels that is distinct from the locations of abnormal tissue voxels. Notably, unlike other detection methods that directly detect a presence of abnormal tissue voxels, or rely on clustering of abnormal tissue voxels, the present example methods do not generally require or rely on clustering of voxels associated with abnormal tissue and instead benefit from a suitably high degree of clustering of the normal tissue voxels. For example, when tumor is the desired abnormal tissue for detection, direct detection methods have difficulty identifying the cluster representative of the tumor because of the inherent heterogeneity of the tumor. Suitable imaging modalities and associated parameters are ones where the resulting normal tissue parameter space region can be defined by a clustering algorithm and that the intersection of this normal tissue cluster and abnormal tissue voxels is zero or close to zero.

It will be understood that clustering of normal tissue voxels and the overlap of the voxels associated with the normal tissue distribution with abnormal tissue voxels may vary depending on a wide variety of factors, including, but not limited to, the choice of imaging modalities, system acquisition parameters employed to generate the imaging datasets, and the pathology or set of pathologies associated with abnormal tissue for the anatomical region of interest. The skilled artisan will be able to experiment with different combinations of system acquisition parameters of a given imaging modality, different image parameters, or combinations of different imaging modalities, in order to achieve a desired level of clustering of normal tissue and separation between the normal tissue parameter space region and the abnormal tissue voxels that reside beyond the normal tissue parameter space region.

In some example implementations, at least two of the plurality of image datasets are multiparametric image datasets that relate to a common imaging modality. In some example implementations, all of the plurality of image datasets are multiparametric image datasets that relate to a common imaging modality.

In some example implementations, at least two of the plurality of image datasets may relate to different imaging modalities. In some example implementations, all of the plurality of image datasets may relate to different imaging modalities.

In some example implementations, at least two of the parameters are image parameters. In some example implementations, all of the parameters are image parameters. Notably, the present inventors have encountered the surprising result that it is possible to perform abnormal tissue detection and segmentation when employed imaging datasets based on image parameters such as signal intensities (or measures obtained from processing detected signals), without requiring imaging datasets based on intrinsic physical tissue parameters. For example, as will be shown below, the present inventors have been able to achieve accurate detection and segmentation of brain metastases based on multiparametric MRI imaging datasets that yield voxel maps of respective image parameters, without requiring the use of imaging datasets that employ intrinsic physical tissue parameters obtained from quantitative MRI or MRI-fingerprinting. Accordingly, the example embodiments that employ image parameters (either from multiparametric and/or multimodal imaging datasets) may facilitate the robust detection and segmentation of abnormal tissue without requiring complex acquisition methods and without computationally intensive methods that are typically associated with imaging datasets characterizing intrinsic physical tissue parameters.

In some example implementations, at least two of the parameters are intrinsic physical tissue parameters. In some example implementations, all of the parameters are intrinsic physical tissue parameters. In some example implementations, at least one of the parameters is an image parameter and at least one of the parameters is an intrinsic physical tissue parameter.

In some example implementations, at least one parameter is a quantitative magnetic resonance image parameter. In some example implementations, at least one parameter is an intrinsic physical tissue parameter obtained from MRI fingerprinting.

In some example implementations, at least one of the image parameters is a signal intensity, such as, for example, the signal intensity on T1 post-gadolinium or T2 FLAIR image. In some example implementations, at least two image parameters are different signal intensities.

In some example implementations, at least one of the parameters may be generated from two or more other parameters. For example, in some example embodiments, one or more parameters may be generated by reducing a higher-dimensional parameter space to a modified parameter space having a reduced number of dimensions (e.g. via a dimensionality reduction method such as principal component analysis).

In some example implementations, at least two image datasets are normalized. Normalization may be beneficial for image parameters obtained as signal intensities in medical images which may vary between acquisitions, patients or scanners. For example, the signal intensities on T1 weighted, T2 weighted or T2 FLAIR MR images. Normalization is less important when the imaging data is based on an intrinsic physical tissue parameter such as apparent diffusion coefficient (ADC) or relaxation time constants such as T1 or T2.

In some example implementations, at least two of the image datasets pertain to different MRI pulse sequences (multiparametric MRI). For example, in the experience of the inventors, the range of signal intensities for grey/white matter (normal tissue) on T1 MPRAGE post-gadolinium and T2 FLAIR show good clustering of normal tissue and distinct from pathology such as enhancing tumor or edema.

In some example implementations, the image datasets pertain to different CT scan voltages. The different CT scan voltages may be selected to facilitate clustering of normal tissue voxels and a separation of voxels associated with acute hematoma from a normal tissue cluster and associated distribution, for example, to facilitate detection of a brain hemorrhage on dual energy CT (DECT). In this example, two image parameters may be attenuation of tissue at a low and a high kVp. In such a case, normal brain tissue would cluster separately from an acute hematoma given the differences in attenuation coefficients at low and high kVp.

Another non-limiting example involves the detection and segmenting of metastases to bone. In such an example case, the suitable image parameters may be signal intensity on T2 STIR and T1 pre-gadolinium sequences. Normal bone marrow shows low signal intensity on T2 STIR and high signal intensity on T1 whereas bone metastases often show the opposite behavior.

Another non-limiting example involves the detection of malignancy (primary and metastatic adenopathy) on whole body PET/MRI. In this example case, the suitable image parameters may be signal intensity on whole body diffusion weighted imaging (DWI) MRI and standard uptake value (SUV) on PET. Most malignant tissue will demonstrate higher signal intensity on DWI and higher SUV on PET whereas most normal tissue (aside from brain) will show the opposite.

As described above, the subset of voxels identified as being abnormal may be determined (obtained) by the application of parameter space criteria generated based on properties of the normal tissue distribution, such as thresholds or regions of interest in parameter space generated based on properties of the normal tissue distribution, that restrict the subset of voxels identified as being abnormal tissue. Such parameter space criteria may be applied in a manually, automated, or semi-automated basis, and can be applied in a single step, or may be applied as a second step after having first employed a normal tissue parameter space region to obtain an initial subset of voxels identified as being abnormal.

Some non-limiting examples of the application of parameter space criteria are illustrated in FIGS. 4A and 4B. In FIG. 4A, parameter space criteria are applied in the form of (shown at 300 and 310), such that only voxels satisfying these criteria, and residing beyond criteria associated with a normal tissue parameter space region (not shown in the figure), will be associated with abnormal tissue. FIG. 4B shows an alternative example implementation in which additional parameter space criteria is provided in the form of a bounding region 320 that is expected to encompass voxels associated with abnormal tissue. Such additional parameter space criteria may be useful in excluding, in addition to voxels associated with normal tissue, additional voxels that are associated with normal structures such as cerebral spinal fluid (CSF) or blood vessels. An example of the use of additional criteria, applied within the parameter space, that further restricts the subset of voxels identified as being abnormal tissue, is illustrated in the examples below.

While FIGS. 4A and 4B illustrate the application of additional parameter space criteria, in addition to initial parameter space criteria defining a normal tissue parameter space region (based on ellipsoid 220 of FIG. 3A), in other example implementations, parameter space criteria associated with one or more axes of parameter space, being defined based on one or more statistical measures associated with the normal tissue distribution, may be applied to identify abnormal tissue voxels without having first applied initial parameter space criteria enclosing a normal tissue parameter space region. For example, as shown in FIGS. 4C and 4D, criteria (thresholds) associated with the statistical properties of the normal tissue are applied, such that after application, the retained voxels include a portion of the voxels that reside within the initial parameter space criteria defining a normal tissue parameter space region 220 shown in FIG. 3A. Accordingly, the parameter space criteria that is applied to identify abnormal tissue voxels, while being generated based on the statistics of the normal tissue distribution, need not enclose the selected normal tissue cluster.

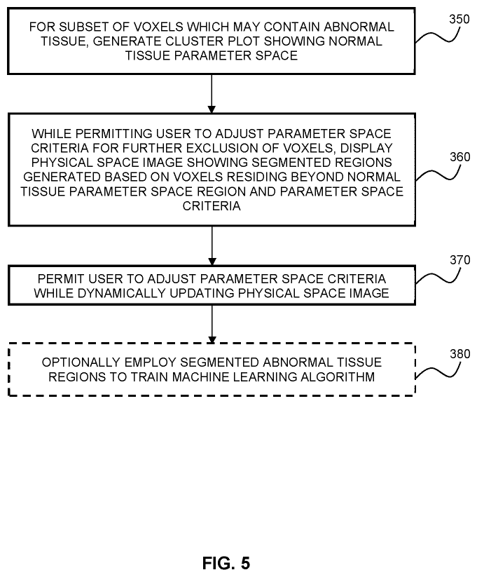

In some example implementations, a user interface may be employed to facilitate the selection and application of the additional parameter space criteria. An example of such an embodiment is illustrated in the flow chart shown in FIG. 5. In step 350, a cluster plot is generated and presented in a user interface. The cluster plot is generated from the imaging datasets based on a subset of voxels that may contain abnormal tissue. As per step 360, the user interface permits the user to apply and vary parameter space criteria, such as the percentiles defining the parameter space boundary associated with a normal tissue parameter space region or the threshold associated with a given parameter or a bounded region of interest within the parameter space used for identifying voxels that are associated with abnormal tissue. For example, the user may adjust the applied criteria by altering percentiles or thresholds in parameter space or manually by editing the region of interest (e.g. erasing or adding voxels with a draw tool). The parameter space criteria can also be modified to include areas of necrosis or expanded to include an additional margin around the normal tissue parameter space region.

In example implementations in which the parameter space is modified from an initial parameter space to a standardized parameter space, based on one or more statistical measures associated with the voxels within the normal tissue parameter space region, thresholds or additional parameter space criteria may be applied within the standardized parameter space, for example, to include or exclude tissues to allow the detection or segmentation of pathology (or normal tissues).

Accordingly, thresholds or additional parameter space criteria may be applied in the parameter space to include or exclude tissues (tissue voxels), for example, using a “points in polygon” computation. The voxels in parameter space that are included or excluded by a threshold or additional parameter space criteria may be projected back to physical space visualize the locations of the voxels.

In some example implementations, a user can select (e.g. click on) the resulting physical space visualization, and connected voxels are used to form the final segmentation. At this stage processing techniques could be applied to exclude or include areas. For example, necrotic areas could be included by using a “filling in” algorithm. Alternatively, the segmentation can be completed by using a region growing algorithm. E.g. the segmentation can be used as a “seed” for a region growing algorithm which can be used to add or remove parts of the segmentation. Alternatively, machine learning (e.g. deep learning) methods may be employed to complete or improve the final segmentation.

As shown in FIG. 5, a physical space image is also rendered, showing the locations of abnormal tissue voxels (and optionally segmented regions) that correspond to voxels that satisfy parameter space criteria (including optional additional user-applied criteria). As shown at step 370, the physical space image is dynamically updated as the user varies the parameter space criteria, thereby enabling the user to dynamically observe the impact of the additional criteria on the identification, distribution and/or segmentation of abnormal tissue regions. As shown at step 380, segmented abnormal tissue regions that are generated based on a final selection of parameter space criteria may be stored and optionally employed for further uses, such as the training of a machine learning algorithm, as described in more detail below. While the preceding example relates to the user-controlled application of additional criteria to restrict voxels that are identified as being associated with abnormal tissue, it will be understood that in other example implementations, additional parameter space criteria may be applied autonomously. In some example implementations, additional criteria may be autonomously applied, for example, when a given set of parameters defining the parameter space are employed (i.e. criteria that is dependent on the choice of parameters), or, for example, based on the selection of a given type of pathology that is to be associated with the identification of abnormal tissue. For example, in some example implementations, an parameter space constraint may be autonomously applied to exclude voxels for which a value of a selected image parameter lies below a threshold determined according to a statistical measure associated with the normal tissue distribution (e.g. three SDs above the average normal tissue value).

In some example implementations, after having segmented a set of abnormal tissue voxels according to one of the example methods disclosed herein (optionally including the aforementioned step of modifying the parameter space according to one or more statistical measures associated with the normal tissue voxels), one or more additional thresholds may be applied to the parameter space representation of the abnormal tissue voxels in order to perform or apply a secondary segmentation to the previously segmented set of abnormal tissue voxels, thereby obtaining a subset of abnormal tissues. This secondary segmentation may include, for example, thresholds in one or more parameter space dimensions. An example of such a secondary segmentation method, as applied to initially segmented abnormal tissue voxels, is described in Example 4 below, in which additional thresholds in parameter space based on T1 Post Gd and T2 FLAIR co-registered images (with the parameter space normalized according to z-score), with additional thresholds applied in each parameter space dimension. Such an approach may be employed, for example, to perform secondary segmentation on the initially segmented abnormal tissue voxels in order to select a subset of abnormal tissue voxels that are deemed to be enhancing tumor.

In some example implementations, after having performed a secondary segmentation on the initially segmented abnormal tissue voxels to obtain a subset of abnormal tissue voxels, the subset of abnormal tissue voxels can be represented within a second parameter space representation that is different from the initial parameter space. The subset of voxels presented in this second parameter space representation (which can be a single or multi-dimensional parameter space) can be processed to identify one or more additional regions of interest. For example, a subset of tumor enhancing voxels, identified via a secondary segmentation as described above, can be represented in a second parameter space (for example, T1MPRAGE post Gd, T2FLAIR post Gd and ADC). The subset of tumor enhancing voxels represented in this second parameter space can be assessed to differentiate, for example, between voxels associated with tumor progression and voxels associated with radiation necrosis. This assessment may be made, for example, at least in part, based on differences in T2 FLAIR post Gd signal and optionally ADC signal. It will be understood that many different sets of imaging modalities can be selected to define the secondary parameter space, and that the thresholds suitable for further voxel typing may be dependent on many factors. For example, an alternative secondary parameter space for differentiating between tumor progression voxels and radiation necrosis voxels may employ T2FLAIR Post Gd and relative cerebral blood volume (CBV).

In the example case in which an initial segmentation of abnormal tissues may include one or more tissues that are desired to be removed, a user may employ a user interface to select a subset of voxels corresponding to the tissue that is to be removed. For example, the user may select voxels for removal using a cursor or other user interface element (e.g. a mouse pointer) to select (e.g. via mouse clicks or keyboard entry) the tissue voxels that are to be removed. In some example implementations, a machine learning algorithm can be employed to suggest a segmented region that corresponds to a given tissue type for removal.

Accordingly, a sampling of the region of tissue that is to be removed can be performed, for example, with a single click, multiple clicks, painting, or other selection methods. The signal intensities (for both p1 and p2 in this example) corresponding to the voxels selected in this process are re-plotted or deleted in parameter space to add or remove parts of the segmentation respectively.

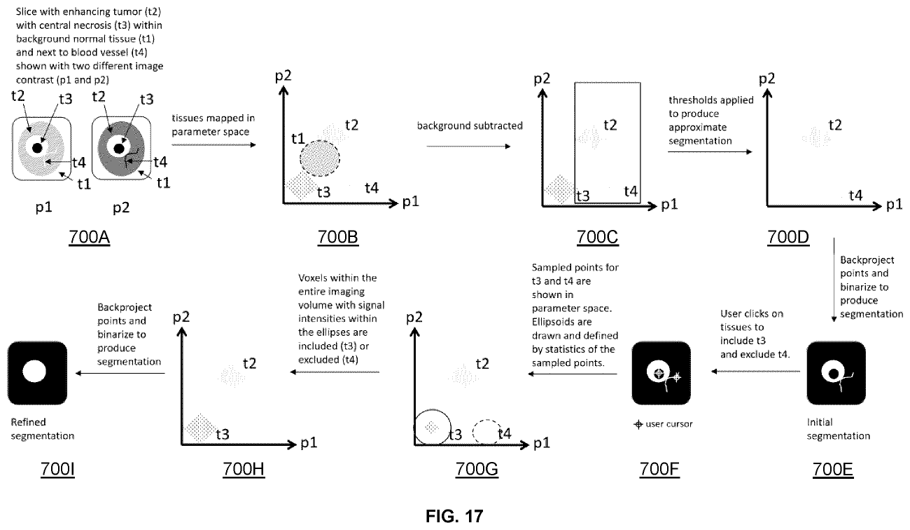

Such a workflow is illustrated in FIG. 17, which shows an initial segmentation of abnormal tissues that includes one or more tissues that are desired to be removed (e.g. adjacent blood vessels or normal tissue) or included (e.g. central area of necrosis). For example, as shown in FIG. 17, an initial segmentation may be performed, using the present example methods involving the identification of a normal tissue parameter space, or based on other parameter space criteria, based on a two-dimensional parameter space involving two different image contrast (p1 and p2). As shown at steps 700A and 700B, the parameter space representation of a given slice may include voxel clusters corresponding to enhancing tumor (t2) with central necrosis (t3) within background normal tissue (t1) and next to blood vessel (t4). As shown at steps 700B, 700C and 700D, in the present example method, the voxels residing within the (previously determined) normal tissue parameter space are subtracted, and additional thresholds are applied to the remaining voxels in order to extract voxels corresponding to the enhancing tumor.

As can be seen at step 700D and after back projection to physical space in step 700E, the threshold that was applied results in the inclusion of a blood vessel. As shown at step 700F, the user identifies, in the physical space image with a cursor or other suitable selection means, voxels corresponding to the central necrosis (t3) for inclusion, and voxels corresponding to the vessel (t4) for exclusion. These sampled points, as identified in the physical image space for inclusion and exclusion, are then shown in parameter space in step 700G, and are processed to generate respective parameter space regions for voxel inclusion and exclusion. These respective regions are then employed, as shown at step 700H, to include and exclude, respective, voxels associated with the central necrosis (t3) and voxels corresponding to vessels (t4) from the entire imaging volume, and the back-projected physical space image is shown in step 7001.

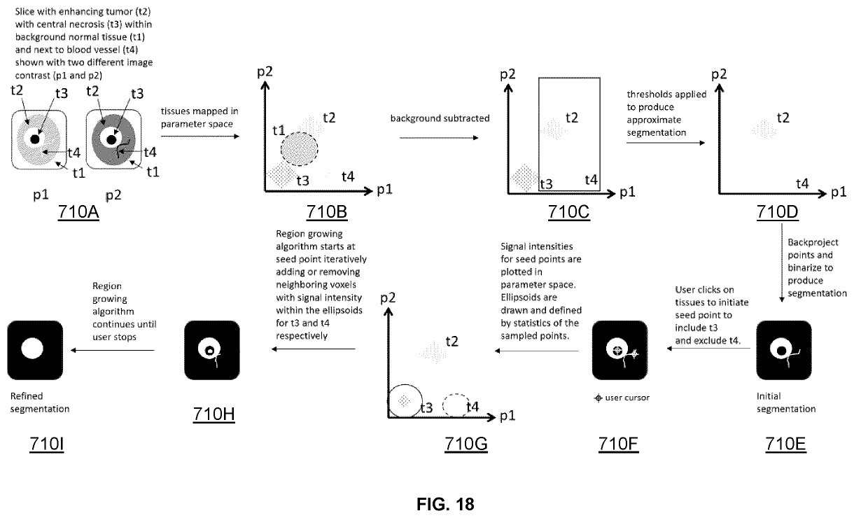

An alternative workflow is illustrated in FIG. 18. While steps 710A-710E are the same as steps 700A-700E of FIG. 17, the voxels selected in step 710F of FIG. 18 are employed to perform a region growing segmentation. The position and signal intensities of the selected region are used as the “seed” points for the algorithm. The region is iteratively grown by evaluating all neighbouring voxels to the region. This can be performed, for example, by measuring the difference between a voxel's signal intensity value on one of the parameters (p1 or p2) and the region's mean on p1 or p2. Voxels with the smallest difference measured this way are allocated to the respective region. This process stops when the intensity difference between region mean and new voxel become larger than a certain threshold. Alternatively, this can be evaluated in a two-dimensional or multidimensional parameter space. Signal intensities from the selected region are plotted in parameter space and the statistics of the region are used to define the respective parameter space regions for voxel inclusion and exclusion, as shown in step 710G. Subsequently, in step 710H, a region growing algorithm starts at a seed point, iteratively adding or removing neighboring voxels with signal intensity within the respective parameter space regions (ellipsoids) for t3 and t4. Neighboring voxels with signal intensities in p1 and p2 that fall within the parameter space regions (ellipses) are included in the region growing segmentation. As the region-growing algorithm proceeds, the segmentation grows or contracts under the supervision of the user. Finally, in step 7101, the region-growing algorithm is terminated once the user decides that the segmentation is completed.

In some example embodiments, the methods disclosed herein may be adapted to produce images with new or hybrid contrast which may be beneficial in diagnosis. Black-blood T1-weighted post gadolinium sequences such as T1 SPACE (sampling perfection with application-optimized contrasts by using different flip angle evolutions) have been shown to improve brain metastasis detection. On these sequences, blood vessels appear dark and brain metastases appear bright which allows for easier detection. However, one of the drawbacks of T1 SPACE is that slow flow within blood vessels can create artifacts which mimic a brain metastasis. While T1 MPRAGE is not subject to these artifacts, blood vessels and brain metastases both appear bright making the latter difficult to detect.

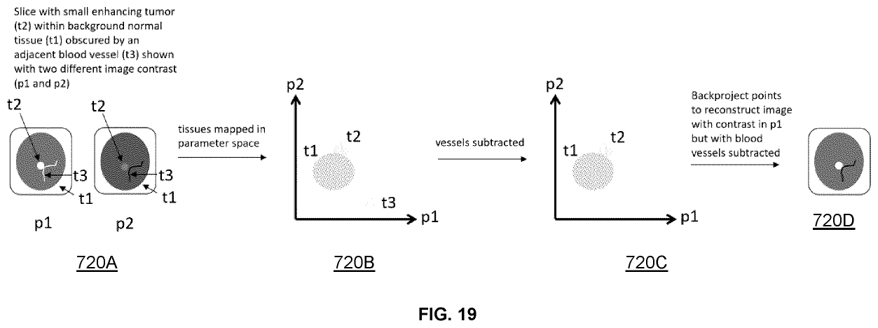

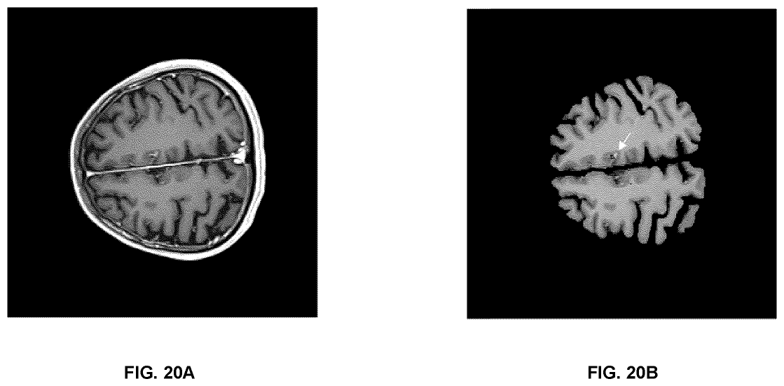

FIGS. 19 and 20 illustrate an example implementation in which blood vessels are removed from a 3D T1 MPRAGE post Gadolinium sequence, resulting in a “pseudo black blood” sequence from a bright blood sequence like T1 MPRAGE, which could improve the sensitivity of these bright blood sequences for metastasis detection without the problem of artifacts.

Referring to FIG. 19, in steps 720A and 720B, a parameter space is created from a bright blood post contrast sequence like T1 MPRAGE (p1) and a commonly acquired T2 weighted sequence such as a fast spin echo T2 FLAIR (p2). Voxels corresponding to normal blood vessels can be identified in the parameter space and removed, as shown in step 720C (i.e. a parameter space region corresponding to the blood vessel tissue can be generated in the parameter space and used to remove these voxels). As shown at step 720D, back projecting the remaining voxels to physical image space results in an image with contrast of p1 but with blood vessels removed. Such an approach can improve the ability to identify brain metastases—essentially turning a T1 weighted bright blood sequence into a black blood sequence.

FIGS. 20A and 20B illustrate how this technique can improve the detectability of small metastases (particularly ones next to blood vessels). The original T1 MPRAGE post Gd image is shown in FIG. 20A, while FIG. 20B shows the blood vessel subtracted image.

It is noted that the subtraction of voxels associated with a given tissue type is but one example of operations that can be performed in the parameter space. Indeed, there are many other operations that can be performed in parameter space or image space. For example, to increase conspicuity, one could move (translate) selected voxels (e.g. voxels belonging to a given tissue type or cluster) within the parameter space, such as further away from the background tissue centroid in parameter space. In image space, this would be equivalent of taking the mask of the voxels and assigning it a value greater or less than one and multiplying the mask by the image, which would have the effect of increasing signal intensity or decreasing signal intensity for voxels within the mask. Alternatively, if one desired to make a structure blend into the background brain (or other anatomical region), the voxels could be moved in parameter space closer to the background tissue centroid. It will be understood that the preceding examples are merely provided to illustrate some non-limiting example methods of performing voxel operations in parameter space to modify the visibility and/or contrast of voxels corresponding to different tissue types, and other operations or modifications may be made without departing from the intended scope of the present disclosure. Furthermore, in some example implementations, a machine learning algorithm (e.g. a deep learning algorithm) could be trained to perform such parameter space operations to modify the visibility of one or more tissue types.

Many of the present example embodiments may be practiced using co-registration of multimodal and/or multiparametric image data acquired at during a single imaging procedure or imaging session. However, it will be understood that in some example implementations, one or more of the image datasets that make up the multimodal and/or multiparametric image dataset may be acquired during different imaging sessions, for example, separated in time by at least one day, one week, or one month.

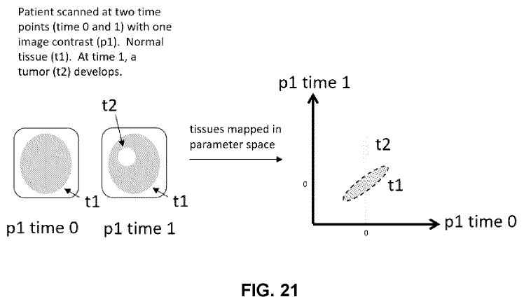

While many of the preceding example embodiments pertain to the use and co-registration of multimodal and/or multiparametric image datasets, where each dimension in parameter space relates to a different imaging parameter, in other example embodiments, at least two dimensions of the parameter space may be associated co-registered imaging data that is acquired at different imaging sessions (e.g. imaging sessions separated in time by at least one day, one week, or one month) and which is associated with the same imaging parameter (e.g. two images of the same contrast acquired at different times). For example, co-registering post Gadolinium T1 data acquired at two different points in time may allow enable the detection of new or growing disease, as these data points would have a different signature in parameter space. For example, with reference to FIG. 21, unchanged voxels will fall along the line of unity in parameter space whereas voxels which have changed between two different points in time will deviate from the line of unity. The changing voxels could then be identified by statistical means as outliers of the linear regression of the unchanging voxels in parameter space.

For example, the prediction interval of the linear regression could be used to determine whether the set of changed voxel intensities would or would not be predicted by the regression.

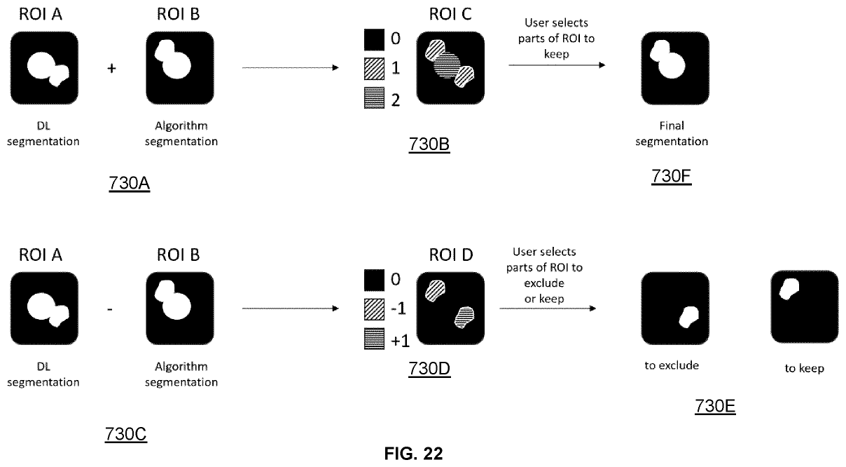

In some example embodiments, the methods of the present disclosure can be adapted for use in tandem with a deep learning algorithm in order assess the accuracy of the deep learning algorithm or to edit the segmentation produced by the deep learning algorithm. An example implementation is illustrated in FIG. 22. A deep learning algorithm produces a first segmentation with resulting ROI A (assigned a value of 1). A user produces a second segmentation using the methods of the present disclosure, resulting in ROI B (assigned a value of 1). The Dice-Sorenson or Hausdorff score for these two segmentations can be calculated and the user can accept the deep learning segmentation or reject it based on these metrics. If rejected, the user can edit the deep learning segmentation, for example, as illustrated in the figure.

As shown in steps 730A and 730B, a new ROI C is created as ROI A+ROI B. A new ROI D is also created as ROI A−ROI B, as shown at steps 730C and 730D, and these masks are attributed different scores. For example, for ROI C, regions with a value 0 are those where both algorithms agree that there is no abnormality, and regions with a value 2 are regions where both algorithms agree that there is an abnormality, while regions with a value of 1 are those where the two algorithms disagree. For ROI D, regions with a value of 0 are those where the two algorithms agree that there is no abnormality or there is an abnormality, regions with a value of −1 are those where the algorithm based on the methods of the present disclosure indicates an abnormality, but the deep learning algorithm does not, and regions with a value 1 are those where the algorithm based on the methods of the present disclosure does not show an abnormality but the deep learning algorithm does indicate an abnormality.

ROI C and ROI D can be displayed to the user, who can select regions to include or exclude in the final selection (as shown in step 730E) for the generation of a final segmentation in step 730F. For example, in the example workflow illustrated in FIG. 22, the region of agreement in ROI C is automatically included in the final segmentation (or selected to be included in the final segmentation), and one or both of the regions of disagreement are selected for inclusion from ROI D, and connected objects (in 2D or 3D) that are selected for inclusion are employed for generation of the final segmentation.

Embodiments of the present disclosure may find use in a wide variety of applications, including for example, the semi-autonomous segmentation of abnormal tissue based on multimodal and/or multiparametric imaging datasets. Indeed, the pathology-agnostic nature of many of the present example embodiments, which, as noted above, employ the properties of normal tissue, as opposed to the properties of pathological tissue, to perform detection and segmentation of abnormal tissue, may be particularly useful in being able to efficiently perform detection and segmentation across a whole organ/imaging volume. Such applications facilitate whole organ screening/detection and segmentation of pathology without any a priori knowledge of the type or location of the pathology in question.

For example, the present methods may find particular utility in facilitating semi-automated pathology segmentation, which currently plays a crucial role in software packages for diagnostic purposes. For example, the present example segmentation methods may be employed for lesion detection, calculating lesion volume and evaluating response to treatment. Segmentations may also be used for treatment planning (e.g. contouring of brain tumors for radiation therapy) and aid in decision making about suitability and method of treatment. Some non-limiting example uses of the present example methods include: liver and lung metastasis detection, segmentation, volume assessment, therapy planning (SBRT) and response to treatment using MRI or CT; prostate cancer detection, segmentation, volume assessment, therapy planning and response to treatment using MRI; and carotid artery vessel wall disease detection, segmentation, volume assessment, and response to treatment using MRI and CT.

Moreover, the initial segmentation of abnormal tissue regions can be an important first step in generating structured datasets to train automated methodologies for detection and segmentation of pathology in medical imaging datasets. For example, the algorithm could be used as an input for other unsupervised or supervised (deep learning) algorithms to complete or refine the segmentation. For example, the algorithm can provide a “seed” for a region growing algorithm. Manual segmentation, while considered the gold standard, is laborious and requires image interpretation expertise. Semiautomated methods are therefore valuable in accelerating the creation of training datasets to be used in automated methods such as machine learning methods that employ deep learning. While some example implementations of the present embodiments may involve the use of segmentation to provide initial data for training of machine learning algorithms, some implementations may involve the use of segmented abnormal tissue regions as input to an already trained algorithm.

In other example applications, abnormal tissue regions segmented according to the present example embodiments may find utility in radiomics and/or radiogenomics applications. For example, radiomics features can be useful for lesion characterization which can aid in pathology specific diagnosis or be used to predict response to treatment or toxicity.

Rapid and easy segmentation will aid the radiologist by streamlining the assessment of changes in disease burden during treatment. It is also expected that the segmentation algorithm will facilitate the treatment of cancer using adaptive radiotherapy by decreasing the time and effort required to segment/re-segment tumors over the course of treatment.

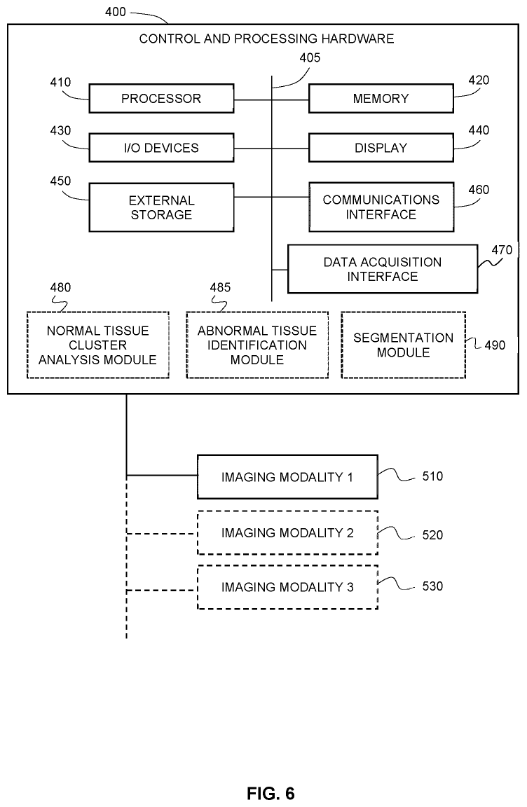

Referring now to FIG. 6, an example imaging system is illustrated for detecting abnormal tissue based on the processing of multiparametric and/or multimodal image datasets. The example system includes control and processing circuitry 400 which is capable of processing imaging datasets obtained from one or more imaging modality subsystems 510-520 (non-limiting examples of which include an MRI system, a PET system, an ultrasound imaging system, and a CT system). In some example embodiments, control and processing hardware 400 may be operably coupled to one or more of the imaging modality subsystems 510-530 (or additional imaging modality subsystems) to control acquisition of imaging datasets. In example implementations in which multiparametric imaging datasets are obtained from a common imaging modality, the imaging datasets may be obtained from a single imaging modality subsystem 510.

As shown in FIG. 6, in one embodiment, control and processing hardware 300 may include a processor 410, a memory 420, a system bus 405, one or more input/output devices 430, and a plurality of optional additional devices such as communications interface 460, display 440, external storage 450, and data acquisition interface 470.

The present example methods can be implemented via processor 410 and/or memory 420. As shown in FIG. 6, the example methods described above, or variations thereof, may be implemented by control and processing hardware 400, via executable instructions represented as normal tissue cluster analysis module 480, abnormal tissue identification module 485 and segmentation module 490.

The functionalities described herein can be partially implemented via hardware logic in processor 410 and partially using the instructions stored in memory 420. Some embodiments may be implemented using processor 410 without additional instructions stored in memory 420. Some embodiments are implemented using the instructions stored in memory 420 for execution by one or more general purpose microprocessors. In some example embodiments, customized processors, such as application specific integrated circuits (ASIC) or field programmable gate array (FPGA), may be employed. Thus, the disclosure is not limited to a specific configuration of hardware and/or software.

Referring again to FIG. 6, it is to be understood that the example system shown in the figure is not intended to be limited to the components that may be employed in a given implementation. For example, the system may include one or more additional processors. Furthermore, one or more components of control and processing hardware 400 may be provided as an external component that is interfaced to a processing device.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed herein can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Example Workflow for Identification and Segmentation of Brain Metastases

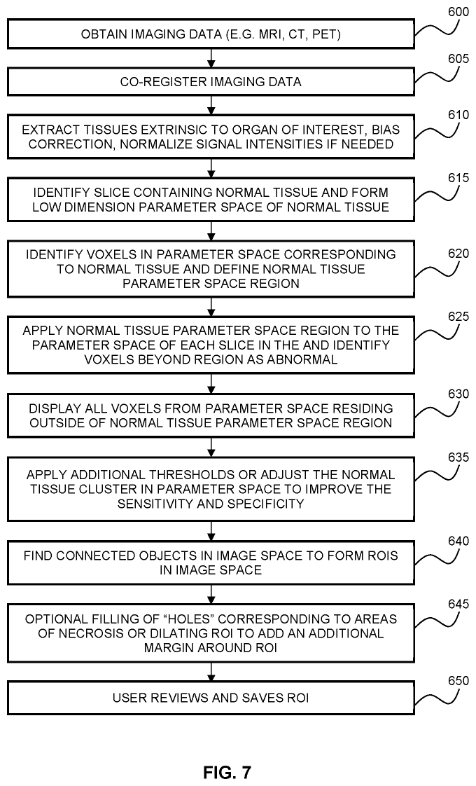

The present example illustrates the non-limiting application of the aforementioned methodology to the detection and segmentation of brain metastases, as illustrated in the flow chart provided in FIG. 7. The imaging datasets were obtained using MR of the brain, collecting multiparametric MRI datasets including diffusion weighted images (DWI, b=1000, RESOLVE technique), 3D T1 MPRAGE post gadolinium, Ax T2 FLAIR post gadolinium (slice thickness=3-5 mm), as shown at 600. The multiparametric image datasets were co-registered using a rigid 6 degree of freedom general registration algorithm (BRAINS) using 3D Slicer (slicer. org) as shown at 605. The brain extraction was then accomplished in step 610 using the b=1000 DWI dataset to create a binary mask of the brain using an arbitrary threshold but any brain extraction algorithm will suffice. The brain extracted 3D T1 MPRAGE and Ax T2 FLAIR datasets were bias corrected using a standard methodology (N4ITK) using 3D Slicer (slicer.org) and the voxel signal intensities were rescaled from 0-1. The upper or lower values of the signal intensities were not excluded, although this could step could be optionally included.

A slice of normal brain was manually identified, as per step 615. The parameter space, generated based on the signal intensity on 3D T1 MPRAGE versus T2 FLAIR, was plotted for this normal tissue slice. As shown at step 620, the normal tissue cluster comprising the brain parenchyma (white matter and grey matter) in the parameter space was then identified using k-means clustering and a region of interest (ROI), that is, the normal tissue parameter space region, was defined using an ellipsoid with center corresponding to the mean signal intensities and axes corresponding to 2 standard deviations in each of the image parameter directions.

The ROI corresponding to normal tissue in parameter space was then applied to the entire brain volume and used to remove the normal voxels from each slice in the imaging volume, as shown in step 625. The remaining voxels displayed in image space will show the brain metastases, as shown at step 630. To alter the appearance of the brain metastases and to improve the sensitivity and specificity of the algorithm, additional thresholds can be applied along at least one of the image parameter space dimensions, as shown at step 635.

Groups of connected voxels/objects in image space are then found in the imaging volume, as shown at step 640, thereby segmenting regions of abnormal tissue. As shown at step 645, “holes” corresponding to areas of necrosis may be optionally filled, and/or the ROI corresponding to the normal tissue parameter space may be dilated to add an additional margin. Finally, as shown at step 650, the user selects and saves ROIs corresponding to brain metastases.

The sample output for the detection algorithm is shown in FIG. 8. The first panel shows T1 MPRAGE post gadolinium source images. The middle panel shows an overlay of metastases detected using the algorithm. The gold standard, manual segmentation, is shown on the far right panels. It is noted that the algorithm is able to distinguish between a brain metastases and transverse venous sinus (white arrows) which appear similar in signal intensity on the T1 MPRAGE post gadolinium source image.

### Example 2: Application of Additional Parameter Space Criteria to Further Restrict Voxels Identified as Being Associated with Abnormal Tissue

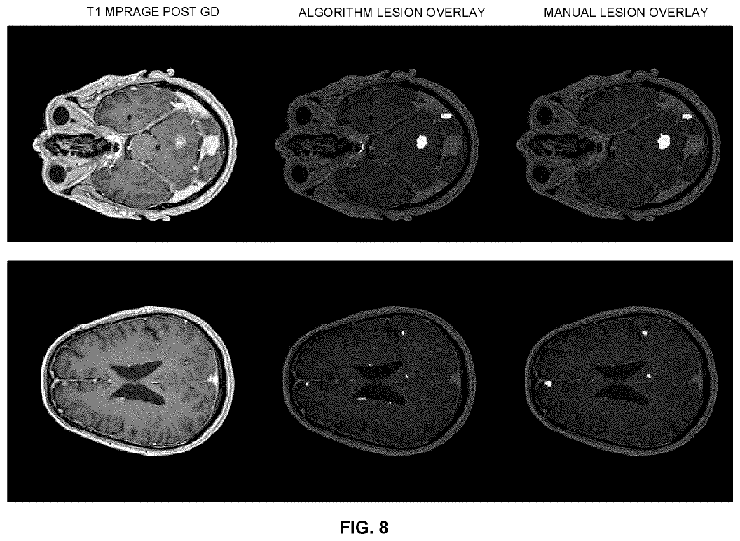

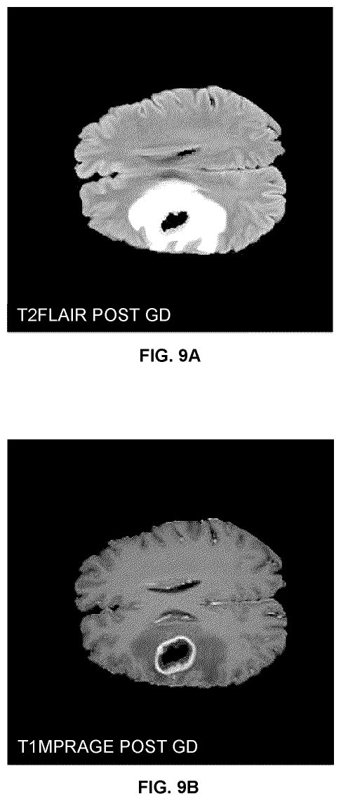

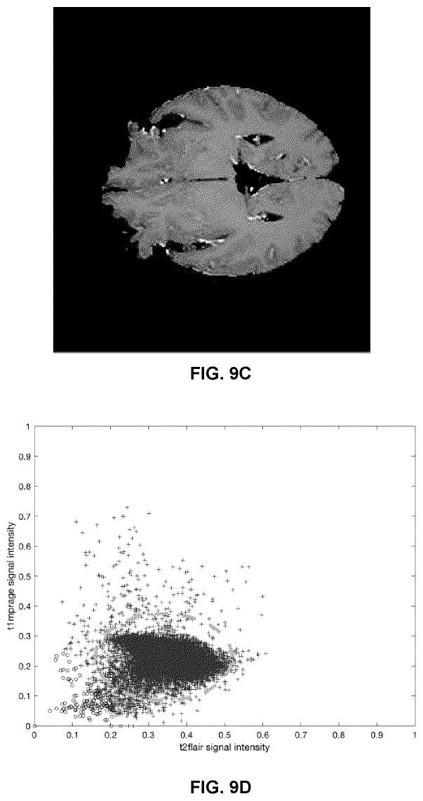

This example illustrates the use of varying thresholds to modify the specificity and sensitivity of lesion detection. FIGS. 9A and 9B show co-registered MR data for two different pulse sequences, namely T2FLAIR post-gad and T1MPRAGE post-gad. FIG. 9C shows a brain slice that is absent of brain metastases that was selected for the initial definition of the normal tissue parameter space.

A cluster (dot) plot for this slice, within the two-dimensional parameter space associated with the parameters of the imaging datasets, is shown in FIG. 9D. K-means clustering was used to define the normal brain voxels (crossed) and an ellipsoid ROI with axes defined by mean signal intensity +/−3SD for the two different image contrast was defined as the normal tissue parameter space region (dashed ellipsoid). The circles in the lower-left corner of the figure represent CSF.

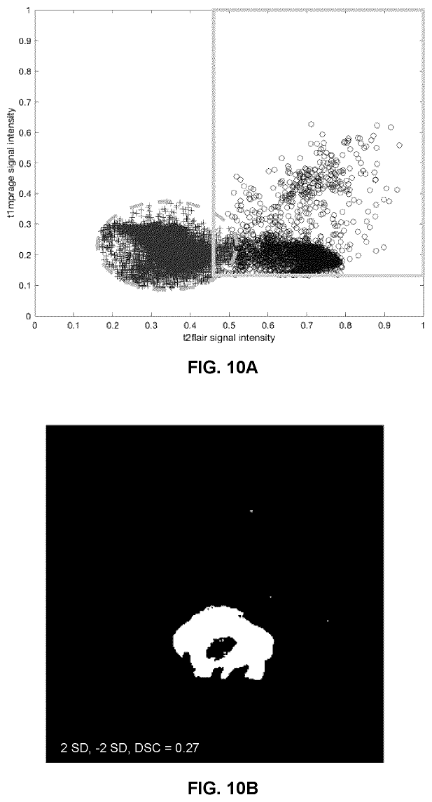

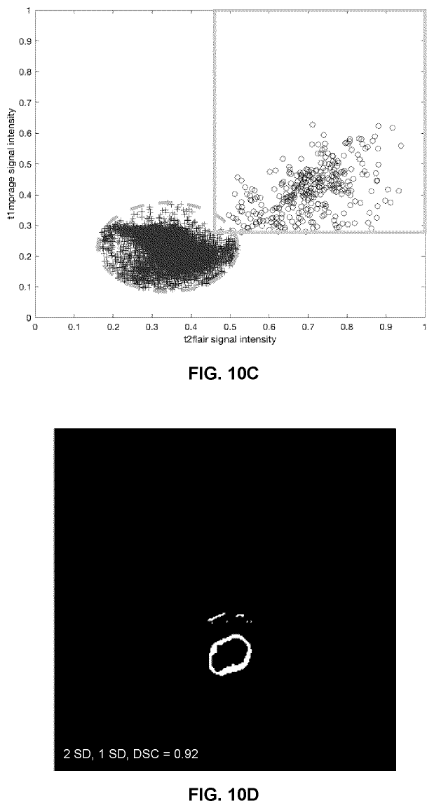

FIG. 10A shows a parameter space cluster for another slice (different from the normal tissue slice). As can be seen in the cluster plot, additional voxels, shown as circles, reside outside of the dashed ellipsoid defining the normal tissue parameter space region, indicating the presence of brain metastases. The ellipsoid ROI defined on the normal brain slice was applied onto the slice containing the brain metastasis (dashed) to remove normal brain voxels (crosses).

FIG. 10A also shows the application of additional parameter space thresholds. As can be seen in the figure, the voxels associated with abnormal tissue were further restricted as those residing above minimum thresholds of each parameter. A physical space image was generated during this process, showing segmented regions of the voxels identified as being associated with abnormal tissue, to permit the operator to dynamically view the impact of different thresholds on the tissue region identified as being abnormal while modifying the thresholds.

In FIG. 10A, a lower threshold set at −2 SD from the mean of the normal tissue cluster distribution on T1 MPRAGE (z-score=−2) was used. The corresponding physical space image of the segmented abnormal tissue shown in FIG. 10B, showing how both the enhancing and non-enhancing (T2 FLAIR hyperintense=edema) part of the tumor can be segmented. FIG. 10C shows another example case in which a higher threshold set at +2 SD from the mean of the normal tissue distribution cluster excludes lower T1 MPRAGE signal intensities was used. The corresponding physical space image of the segmented abnormal tissue shown in FIG. 10D, showing how this threshold results in the selection of only the enhancing part of the tumor.

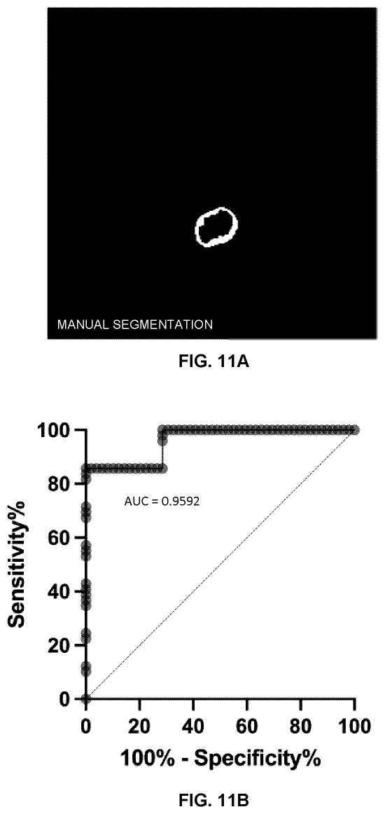

FIG. 11A shows the result of lesion detection using the gold standard of manual segmentation of the entire brain tumor. FIG. 11B shows the performance of the present example implementation of the normal-tissue-based segmentation algorithm, generated using 49 different combinations (ranging from mean−3SD to mean+3SD for T2 FLAIR and T1 MPRAGE signal intensities), showing the resulting ROC analysis. As can be seen from the figure, the present example method had a high performance relative to the gold standard, exhibiting an AUC of 0.96.

### Example 3: Isolation of Metastases Relative to Blood Vessels

The present example demonstrates the adaptation of the previously described example embodiments for segmenting enhancing brain lesions while also excluding blood vessels, which may be abutting or traversing a lesion of interest. The presence of such structural features can be a problem for existing semiautomatic segmentation algorithms that utilize thresholding on a single imaging sequence such as T1 post gadolinium.

In the present example implementation, the multiparameter MRI imaging datasets were selected as 3D T1 MPRAGE post gadolinium and an axial T2 FLAIR sequence. Imaging datasets with these MRI image parameters are particularly useful for blood vessel segmentation because flowing blood on the T2 FLAIR sequence is low signal intensity (black blood) and generates a high uniform signal on 3D T1 MPRAGE post gadolinium sequence. Brain metastases may have similar signal intensity on 3D T1 MPRAGE post gadolinium but will have a higher signal intensity than blood pool on T2 FLAIR sequences. By adjusting the threshold on the T2 FLAIR dimension of the parameter space, more or less blood vessel can be included in the segmentation. While a 2D T2 FLAIR sequence was utilized, the methodology is expected to also facilitate segmentation for most other black blood 2D or 3D sequences.

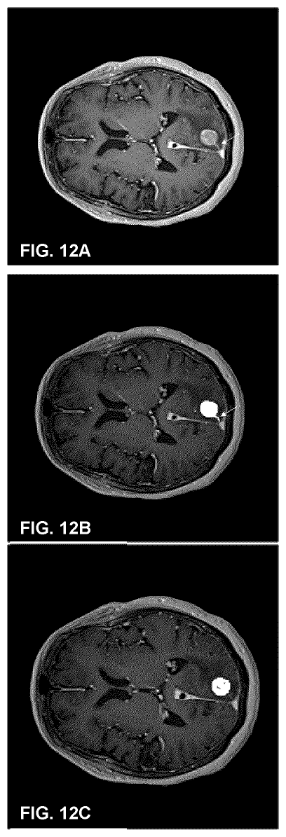

FIGS. 12A-12C demonstrate blood vessel exclusion on tumor segmentation using the present example method. FIG. 12A shows a solitary occipital metastasis with small vessel abutting the metastasis (white arrow) on 3D T1 MPRAGE. In FIG. 12B, brain tumor segmentation is performed using thresholds of mean signal intensity of T2 FLAIR of the normal brain cluster, resulting in inclusion of the blood vessel (white arrow). In contrast, in FIG. 12C, brain tumor segmentation using threshold of signal intensity +3 standard deviation above mean T2 FLAIR signal intensity of the normal brain cluster shows removal of the blood vessel in the segmentation.

### Example 4: Segmentation With Parameter Space Normalized Based on Statistical Measures Associated with Normal Tissue

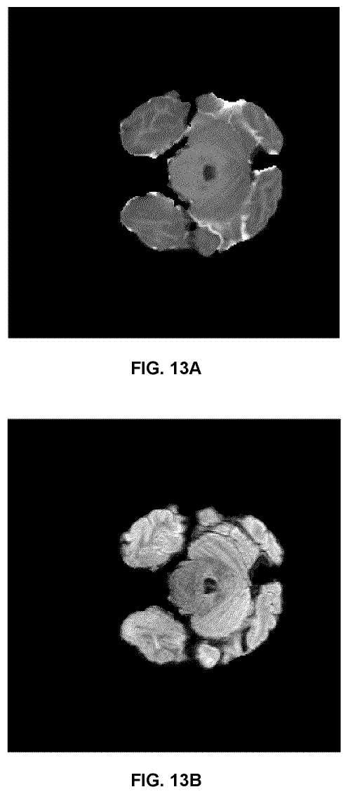

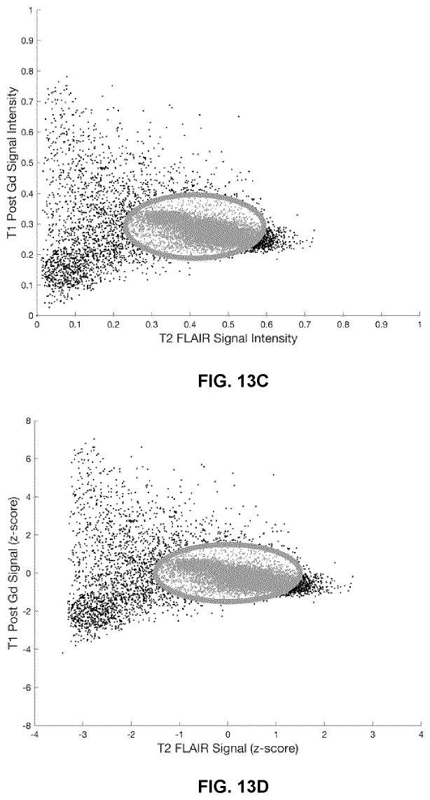

The present example describes a workflow in which segmentation of abnormal tissue voxels is performed based on a parameter space that is normalized (modified) according to statistical measures associated with the normal tissue. FIGS. 13A and 13B show rescaled signal intensities of T1 MPRAGE and T2 FLAIR, respectively, from a slice of normal brain. These rescaled images are used to create an initial parameter space, shown in FIG. 13C. K-means clustering is used to identify signal intensities corresponding to normal grey and white matter (e.g. a “background layer”) which can be approximated by an ellipsoid (grey) with a center corresponding to the centroid of the ellipse. In the example shown, the semi-axes lengths of the ellipse were set to 1.5 SD of signal intensities for each axis. To define the normalized parameter space, the centroid of the background layer cluster was used to define the origin of the parameter space and the axes units are defined by the z-score of signal intensities, with the resulting normalized parameter space shown in FIG. 13D.

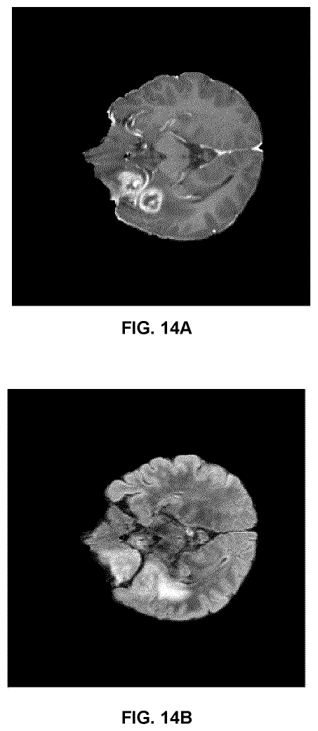

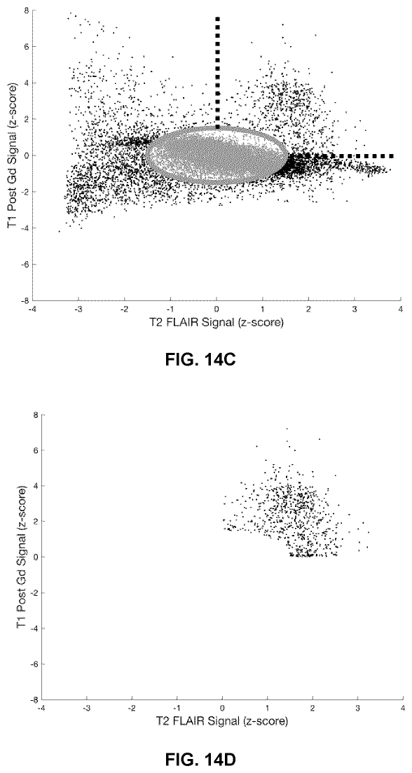

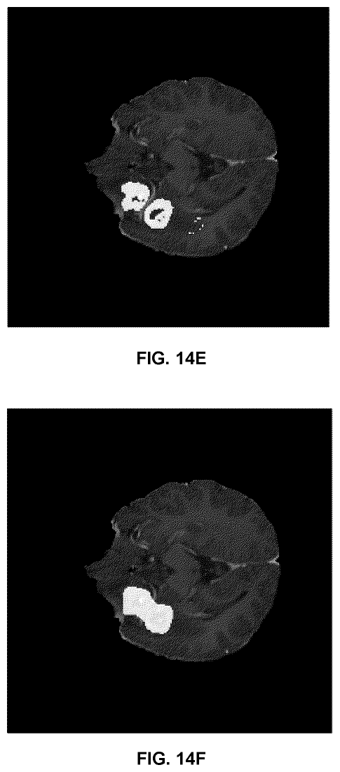

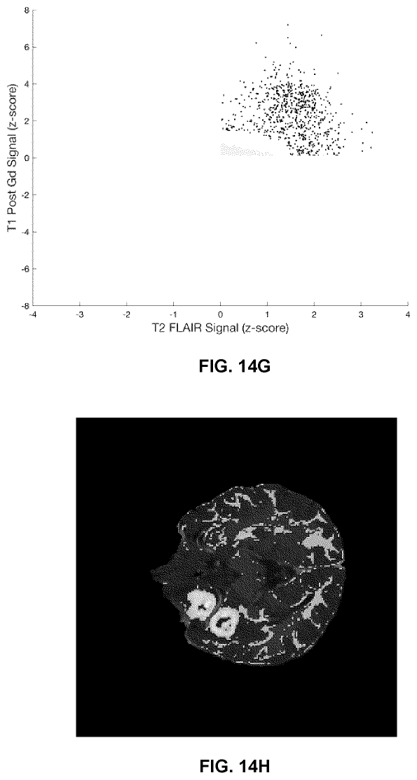

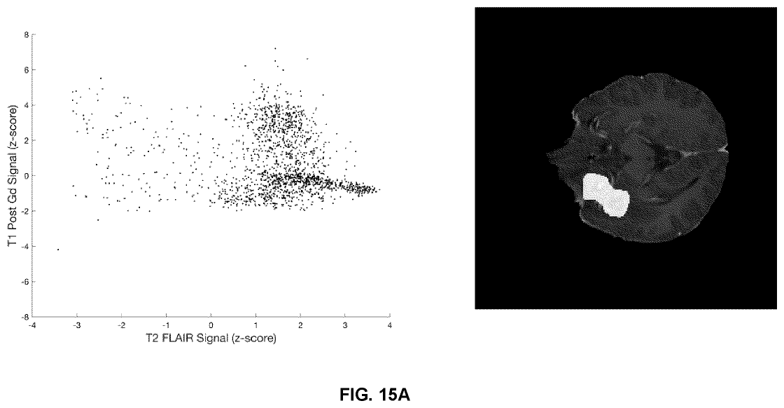

Segmentations were then performed using the normalized parameter space. FIGS. 14A and 14B show the rescaled signal intensities of T1 MPRAGE and T2 FLAIR, respectively, from a slice containing pathology (a brain tumor in this example). These images were re-expressed in the parameter space that was normalized according to the statistical measures associated with the normal slice of brain, as shown in FIG. 14C.

Segmentations were performed by subtracting the background layer ellipsoid (grey ellipse) and applying thresholds set at the mean of the normal tissue distribution (dashed line, z-score=0) in T1 MPRAGE and T2 FLAIR, as shown in FIG. 14C. The remaining voxels, as shown in FIG. 14D, in the parameter space defined by the normal tissue statistics, are projected back into image space to visualize the resulting segmentation of enhancing tumor, as shown in FIG. 14E.

A segmentation of the whole tumor (enhancing tumor plus non-enhancing tumor) was performed by selecting the 3D object consisting of connected enhancing voxels and filling in non-enhancing or necrotic regions utilizing morphological dilatations and erosion with structuring elements, resulting in the image shown in FIG. 14F.

FIGS. 14G and 14H show application of thresholds set at the mean of the normal tissue distribution in both T1 MPRAGE and T2 FLAIR (z-score=0). The background layer ellipsoid was not subtracted in this case leaving behind voxels corresponding to normal brain (light grey). Back projecting all the voxels in parameter space produces a segmentation which includes both tumor and normal brain (FIG. 14H).

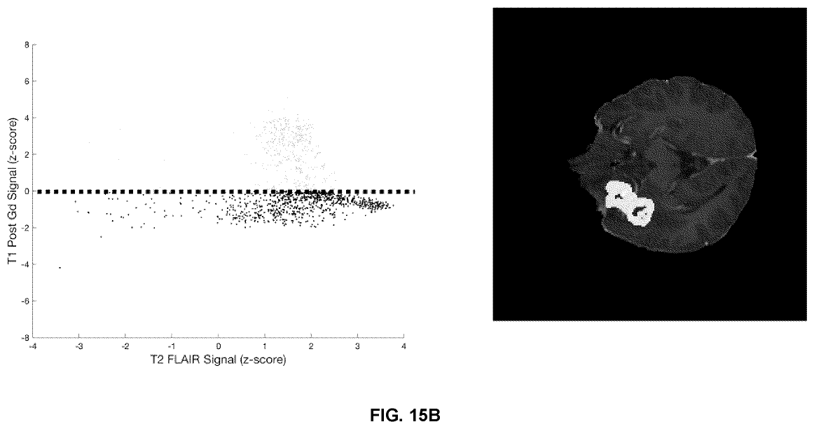

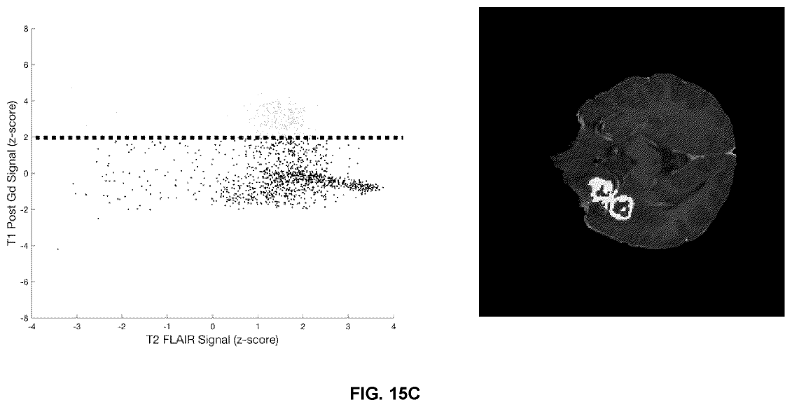

Referring now to FIGS. 15A-15C, segmentations of the enhancing tumor were re-derived from segmentations of whole tumor by applying different T1 MPRAGE thresholds in the normalized parameter space described above. FIG. 15A shows the enhancing tumor voxels in the normalized parameter space (left) and the associated image-space projection of these voxels (right). FIGS. 15B and 15C show the application of thresholds to the enhancing tumor voxels (left), with a T1 MPRAGE threshold set to z-score of 0 and 2, respectively. The figures show segmentations on the right, in which voxels with signal intensities below the threshold value in the normalized parameter space (black) are excluded and the remaining voxels (gray) are retained to visualize the segmentation. Deriving segmentations of enhancing tumor in this way can therefore be standardized which can improve reproducibility of the segmentation.

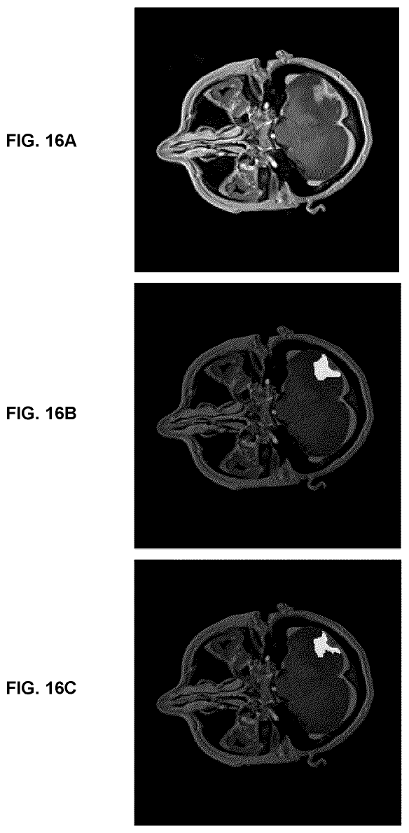

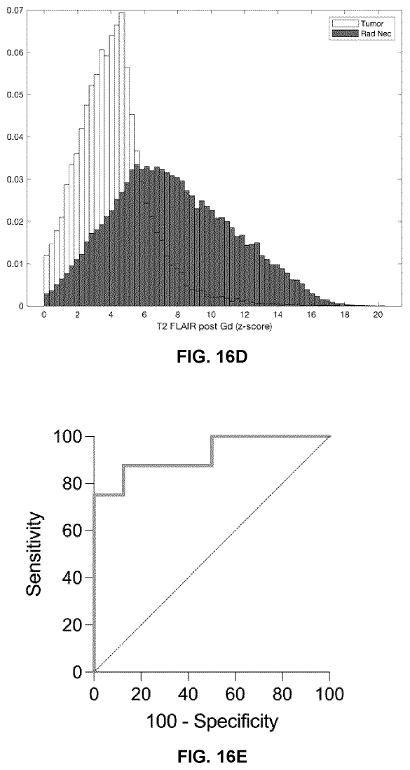

FIG. 16A shows the rescaled signal intensities of T1 MPRAGE post Gadolinium for a patient who had prior radiation to the cerebellum for metastatic disease and now presents with increasing enhancement. A segmentation of the whole tumor (enhancing tumor plus non-enhancing tumor) was performed as described previously, with the resulting image-space projection shown in FIG. 16B. As described previously, the segmentation of the whole tumor was used as a starting point to produce a segmentation of enhancing tumor using a T1 MPRAGE threshold of 0 z-score, with the resulting image-space projection shown in FIG. 16C.

3D ROIs of enhancing tumor were produced for 16 patients with previous radiation for brain metastases with follow-up or pathology showing tumor recurrence or radiation necrosis. The ROIs were overlayed on co-registered T2 FLAIR post Gadolinium images and the normalized signal intensities from all voxels within the ROIs were extracted for all tumors and all patients with either tumor progression (white) or radiation necrosis (dark gray), as shown in FIG. 16D. As can be seen from the figure, patients with radiation necrosis show higher normalized T2 FLAIR post Gadolinium signal intensities. Accordingly, using a cut-off of T2 FLAIR post Gadolinium signal intensity (z-score) of 4.5, an ROC analysis was performed, as shown in FIG. 16E which resulted in an AUC=0.92.

### Example 5: Tracking of Tumor Progression

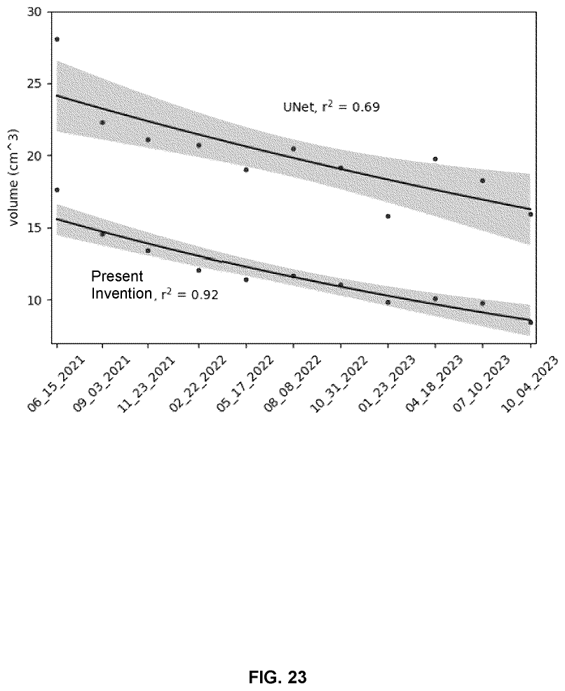

FIG. 23 shows non-enhancing tumor volume measurements for a patient with a low grade glioma on targeted treatment. Tumor volumes were measured using segmentation performed with an nnUnet as well as the present invention and fitted to an exponential (95% Cl shown). Both methodologies show a progressive decrease in tumor volume with treatment but the nnUnet tends to overestimate tumor volume and shows a poorer fit compared to the method of the present invention.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of processing image data to identify abnormal tissue, the method comprising:
obtaining a plurality of co-registered image datasets associated with a subject, each image dataset associating values of a respective image parameter with a set of voxels, the respective image parameters of the plurality of co-registered image datasets defining a parameter space;
selecting voxels associated with normal tissue;
determining properties of a distribution associated with the selected normal tissue voxels;
employing properties of the distribution to determine parameter space criteria suitable for identifying abnormal tissue voxels; and
processing the image datasets to identify, from a selected set of voxels, an abnormal set of voxels having image parameters satisfying the parameter space criteria.

2. The method according to claim 1 further comprising generating an image that facilitates identification, in physical space, of locations at least a portion of the abnormal set of voxels.

3. The method according to claim 2 further comprising defining, within the parameter space, a normal tissue parameter space region based on statistical properties of the distribution.

4. The method according to claim 3 wherein the parameter space criteria is generated based on the normal tissue parameter space region.

5. The method according to claim 1 wherein the parameter space criteria comprises one or more thresholds, each threshold being associated with a respective parameter space axis, and wherein each threshold is generated based on a statistical measure associated with the distribution.

6. The method according to claim 1 wherein the parameter space criteria comprises a region having boundary values determined based on statistical measures associated with the distribution.

7. The method according to claim 3 wherein the image includes normal voxels having image parameters residing within the normal tissue parameter space region, and wherein the abnormal set of voxels are displayed such that they are identifiable relative to the normal voxels.

8. The method according to claim 3 wherein the image excludes normal voxels having image parameters residing within the normal tissue parameter space region.

9. The method according to claim 1 further comprising employing a segmentation algorithm to segment at least one contiguous group of voxels within the abnormal set of voxels, thereby obtaining a segmented region.

10. The method according to claim 9 further comprising generating an image that facilitates identification of the segmented region.

11. The method according to claim 10 further comprising employing the segmented region to train a machine learning algorithm.

12. The method according to claim 11 wherein the segmented region is employed as a seed for a region-growing algorithm.

13. The method according to claim 10 further comprising processing the image to identify a necrotic region and annotating the image within the necrotic region.

14. The method according to claim 1 wherein the abnormal set of voxels are identified from voxels corresponding to a single image slice.

15. The method according to claim 1 wherein the abnormal set of voxels are identified from voxels corresponding to a plurality of image slices.

16. The method according to claim 1 wherein the abnormal set of voxels are identified from voxels spanning an entirety of an organ.

17. The method according to claim 1 wherein the parameter space is modified, prior to identifying the abnormal set of voxels, according to one or more statistical measures associated with the parameter values of voxels associated with normal tissue.

18. The method according to claim 1 wherein at least one parameter space axis of the parameter space is normalized, prior to identifying the abnormal set of voxels, based on a z-score of voxels associated with normal tissue.

19. The method according to claim 1 wherein the parameter space origin is modified, prior to identifying the abnormal set of voxels, based on a centroid associated with voxels associated with normal tissue.

20. The method according to claim 1 wherein the parameter space criteria is autonomously applied.

21. The method according to claim 1 wherein the parameter space criteria is selectable on a user interface presented to a user.

22. The method according to claim 4 further comprising applying additional parameter space criteria, prior to obtaining the abnormal set of voxels from the selected set of voxels, such that the abnormal set of voxels are excluded from the normal tissue parameter space region and also satisfy the additional parameter space criteria.

23. The method according to claim 1 further comprising, after having obtained the abnormal set of voxels, applying additional parameter space criteria to the abnormal set of voxels, thereby obtaining a subset of abnormal voxels that satisfy the additional parameter space criteria.

24. The method according to claim 23 wherein the additional parameter space criteria is configured such that the subset of abnormal voxels corresponds to tumor enhancing voxels.

25. The method according to claim 23 wherein the parameter space is an initial parameter space, the method further comprising representing the subset of abnormal voxels in a secondary parameter space that is different from the initial parameter space.

26. The method according to claim 25 further comprising applying a supplementary criterion to the secondary parameter space to differentiate among at least two abnormal tissue types.

27. The method according to claim 26 wherein the additional parameter space criterion is configured such that the subset of abnormal voxels correspond to enhancing tumor voxels, and wherein the supplementary criterion is configured to facilitate differentiation between voxels associated with tumor progression and voxels associated with radiation necrosis.

28. The method according to claim 27 wherein the supplementary criterion comprises a T2 FLAIR post Gadolinium signal threshold.

29. The method according to claim 28, wherein the parameter space criteria is based on a normal tissue parameter space region defined according to statistical properties of the distribution, the method further comprising:
generating a cluster plot showing:
locations of the selected set of voxels in the parameter space;
the normal tissue parameter space region; and
an annotation representing the additional parameter space criterion;
generating a physical space image showing locations of voxels excluded from the normal tissue parameter space region and satisfying the additional parameter space criterion;
receiving input from the user for adjusting the additional parameter space criterion; and
dynamically updating the physical space image as the additional parameter space criterion is varied by the user.

30. The method according to claim 1 wherein the normal tissue parameter space region is determined by processing the plurality of co-registered image datasets, within the subset of voxels associated with normal tissue, such that the normal tissue parameter space region substantially encloses a cluster of normal tissue voxels.

31. The method according to claim 1 wherein the normal tissue parameter space region is determined by processing reference image data associated with a set of reference subjects.

32. The method according to claim 1 wherein the normal tissue parameter space region is determined, at least in part, according to user input, the user input identifying a subset of the set of voxels that is associated with the presence of normal tissue.

33. The method according to claim 32 wherein the set of voxels spans a single image slice identified as containing normal tissue.

34. The method according to claim 32 wherein the set of voxels spans a plurality of image slices identified as containing normal tissue.

35. The method according to claim 30 further comprising receiving user instructions to dilate the normal tissue parameter space region, and dilating the normal tissue parameter space region according to the user instructions.

36. The method according to claim 1 wherein the set of voxels are separated from a larger set of voxels prior to applying the parameter space criteria, the set of voxels pertaining to an anatomical region of interest that resides within the larger set of voxels.

37. The method according to claim 1 wherein the image parameter associated with at least one of the image datasets is a signal intensity.

38. The method according to claim 1 wherein at least two image datasets of the plurality of co-registered image datasets are associated with different imaging modalities.

39. The method according to claim 1 wherein at least two plurality of co-registered image datasets of the plurality of co-registered image datasets are multiparametric image datasets associated with a common imaging modality.

40. The method according to claim 39 wherein at least two imaging datasets of the multiparametric image datasets are magnetic resonance imaging datasets.

41. The method according to claim 40 wherein the magnetic resonance imaging datasets include a T1 MPRAGE post-gadolinium image dataset and a T2FLAIR post-gadolinium image dataset.

42. The method according to claim 40 wherein the magnetic resonance imaging datasets include a T2 STIR image dataset and a T1 pre-gadolinium image dataset.

43. The method according to claim 39 wherein at least two imaging datasets of the multiparametric image datasets include CT imaging datasets acquired with different scan voltages.

44. The method according to claim 1 wherein at least two of the image datasets are normalized.

45. A system for processing image data to identify abnormal tissue, the system comprising:
processing circuitry comprising at least one processor and associated memory, said memory storing instructions executable by said at least one processor for performing operations comprising:
obtaining a plurality of co-registered image datasets associated with a subject, each image dataset associating values of a respective image parameter with a set of voxels, the respective image parameters of the plurality of co-registered image datasets defining a parameter space;
selecting voxels associated with normal tissue;
determining properties of a distribution associated with the selected normal tissue voxels;
employing properties of the distribution to determine parameter space criteria suitable for identifying abnormal tissue voxels; and
processing the image datasets to identify, from a selected set of voxels, an abnormal set of voxels having image parameters satisfying the parameter space criteria.

46. A non-transitory computer-readable storage medium having stored therein data representing instructions executable by a processor for processing image data to identify pathology, the storage medium comprising instructions for performing operations including:
obtaining a plurality of co-registered image datasets associated with a subject, each image dataset associating values of a respective image parameter with a set of voxels, the respective image parameters of the plurality of co-registered image datasets defining a parameter space;
selecting voxels associated with normal tissue;
determining properties of a distribution associated with the selected normal tissue voxels;
employing properties of the distribution to determine parameter space criteria suitable for identifying abnormal tissue voxels; and
processing the image datasets to identify, from a selected set of voxels, an abnormal set of voxels having image parameters satisfying the parameter space criteria.
